SEQUENCE LISTING (18 sequences)

SEQ ID NO: 1 - protein, 9 aa, Artificial Sequence
  Synthetically generated peptide
GPICFFRLG

SEQ ID NO: 2 - protein, 7 aa, Artificial Sequence
  Synthetically generated peptide
HSSKLQG

SEQ ID NO: 3 - protein, 18 aa, Artificial Sequence
  Synthetically generated peptide
GRKKRRQRRRKGDEVDGC

SEQ ID NO: 4 - protein, 4 aa, Artificial Sequence
  Synthetically generated peptide
RRKC

SEQ ID NO: 5 - protein, 5 aa, Artificial Sequence
  Synthetically generated peptide
GRRKC

SEQ ID NO: 6 - protein, 7 aa, Artificial Sequence
  Synthetically generated peptide
GRRRRKC

SEQ ID NO: 7 - protein, 8 aa, Artificial Sequence
  Synthetically generated peptide
GRRGRRKC

SEQ ID NO: 8 - protein, 11 aa, Artificial Sequence
  Synthetically generated peptide
GFGSVQFAGKC

SEQ ID NO: 9 - protein, 7 aa, Artificial Sequence
  Synthetically generated peptide
GFLGGKC

SEQ ID NO: 10 - protein, 9 aa, Artificial Sequence
  Synthetically generated peptide
GPLGVRGKC

SEQ ID NO: 11 - protein, 11 aa, Artificial Sequence
  Synthetically generated peptide
GFXRSGGGGKC

SEQ ID NO: 12 - protein, 11 aa, Artificial Sequence
  Synthetically generated peptide
GFPRSGGGGKC

SEQ ID NO: 13 - protein, 31 aa, Artificial Sequence
  Synthetically generated peptide
XXCXXXXCXXXFXXXXXLXXHXXXHXXXXXX

SEQ ID NO: 14 - protein, 12 aa, Artificial Sequence
  Synthetically generated peptide
XXXXXXXXXXXX

SEQ ID NO: 15 - protein, 8 aa, Artificial Sequence
  Synthetically generated peptide
GRKKRQRR

SEQ ID NO: 16 - protein, 9 aa, Artificial Sequence
  Synthetically generated peptide
GRKKRRQRR

SEQ ID NO: 17 - protein, 9 aa, Artificial Sequence
  Synthetically generated peptide
GVRLGPGKC

SEQ ID NO: 18 - protein, 11 aa, Artificial Sequence
  Synthetically generated peptide
GFXRPGGGGKC

(19) United States
(12) Patent Application Publication
(10) Pub. No.: US 2005/0169843 A1
(43) Pub. Date: Aug. 4, 2005

(54) Activatable imaging probes

(76) Inventors: Ralph Weissleder, Charlestown, MA (US); Ching-Hsuan Tung, Wayland, MA (US); Umar Mahmood, Winchester, MA (US)

Correspondence Address: FISH & RICHARDSON PC, 225 FRANKLIN ST, BOSTON, MA, 02110, US

(21) Appl. No.: 10/745,336

(22) Filed: Dec. 22, 2003

Related U.S. Application Data
(63) Continuation of application No. 10/039,831, filed on Jan. 4, 2002, now abandoned.
(60) Provisional application No. 60/260,123, filed on Jan. 5, 2001.
(60) Provisional application No. 60/277,352, filed on Mar. 19, 2001.
(60) Provisional application No. 60/346,420, filed on Nov. 9, 2001.

(51) Int. Cl. A61B 10/00

(52) U.S. Cl. 424/9.6

(57) ABSTRACT

The invention relates to activatable imaging probes that includes a chromophore attachment moiety and one or more, e.g., a plurality of, chromophores, such as near-infrared chromophores, chemically linked to the chromophore attachment moiety so that upon activation of the imaging probe the optical properties of the plurality of chromophores are altered. The probe optionally includes protective chains or chromophore spacers, or both. Also disclosed are methods of using the imaging probes for optical imaging.

### CROSS-REFERENCE TO RELATED APPLICATIONS

This application claims priority from the following three United States Provisional Patent Application Ser. No. 60/260,123, filed on Jan. 5, 2001, 60/277,352, filed on Mar. 19, 2001, and Serial Number to be Determined, filed on Nov. 9, 2001, all of which are incorporated herein by reference in their entireties.

### FIELD OF THE INVENTION

The invention relates to biochemistry, cell biology, and optical imaging.

### BACKGROUND OF THE INVENTION

Optically based biomedical imaging techniques have advanced over the past decade due to developments in laser technology, sophisticated reconstruction algorithms, and imaging software originally developed for non-optical, tomographic imaging modes such as CT and MRI. Visible wavelengths are used for optical imaging of surface structures by means of endoscopy and microscopy.

Near infrared wavelengths (approx. 600-1000 nm) have been used in optical imaging of internal tissues, because near infrared radiation exhibits tissue penetration of up to about fifteen centimeters. See, e.g., Wyatt, 1997, “Cerebral oxygenation and haemodynamics in the fetus and newborn infant,” Phil. Trans. R. Soc. London B 352:701-706; and Tromberg et al., 1997, “Non-invasive measurements of breast tissue optical properties using frequency-domain photo migration,” Phil. Trans. R. Soc. London B 352:661-667.

Advantages of near infrared imaging over other currently used clinical imaging techniques include the following: potential for simultaneous use of multiple, distinguishable probes (important in molecular imaging); high temporal resolution (important in functional imaging); high spatial resolution (important in in vivo microscopy); and safety (no ionizing radiation).

In near infrared fluorescence imaging, filtered light or a laser with a defined bandwidth is used as a source of excitation light. The light may be continuous in intensity, pulsed, or may be modulated (for example by frequency or amplitude). The excitation light travels through body tissues (but may remain near the surface, for example at the skin or at an endothelial surface). When the excitation light encounters a near infrared fluorescent molecule (“contrast agent”), the light is absorbed. The fluorescent molecule then emits light that has detectably different properties (i.e., spectral properties of the probe (slightly longer wavelength), e.g., fluorescence) from the excitation light. Despite good penetration of biological tissues by light, conventional near infrared fluorescence probes are subject to many of the same limitations encountered with other contrast agents, including low target/background ratios.

### SUMMARY OF THE INVENTION

The invention is based on the discovery of imaging probes that have altered optical properties after interaction with a target molecule, i.e., activation of the probe. This enables 1) detection of early disease, 2) a high target/background ratio for improved detection of subtle disease, and 3) non-invasive, imaging of internal molecular targets based on their biological activity. The design of the new probes is based on various fluorescence activation strategies, e.g., fluorescence quenching/dequenching, wavelength shifts, polarization, and change in fluorescence lifetime.

One of the major needs facing in vivo molecular imaging is the development of biocompatible molecular beacons that are capable of specifically and accurately measuring in vivo targets at the protein function, protein structure, RNA, or DNA level. The new probes address this need and therefore have widespread applications for real-time in vivo imaging of a variety of clinically relevant targets. For example, the probes can be used to detect endogenous enzyme activity in disease, to monitor efficacy of inhibitors, to help guide surgical interventions, to determine therapeutic doses, and to image gene expression.

In one aspect, the invention features an imaging probe comprising a chromophore attachment moiety and one or more, e.g., a plurality of, chromophores, wherein the chromophores are chemically linked to the chromophore attachment moiety so that upon activation of the imaging probe, the optical properties of the chromophores are altered. In one embodiment, the probe is intramolecularly quenched. In another embodiment, the imaging probe includes one or more quencher molecules that quench the initial signal, wherein dequenching of the chromophores occurs upon activation of the probe. In one embodiment, two separate probes (which may be identical or may have different optical, biological, or chemical properties) become activated when they are in proximity to one another. In these new methods, the probes can be activated by phosphorylation, dephosphorylation, pH mediated cleavage, conformation change, enzyme-mediated splicing, enzyme-mediated transfer of the one or more chromophores, hybridization of a nucleic acid sequence to a complementary target nucleic acid, binding of the probe to an analyte, chemical modification of the chromophore, or binding of the probe to a receptor.

In addition, in these methods, the optical properties of the chromophores can be altered by dequenching, quenching, changes in wavelength, changes in fluorescence lifetime, changes in spectral properties, or changes in polarity or combinations thereof. The chromophores can be fluorochromes, non-fluorescent chromophores, fluorescence quenchers, absorption chromophores, or combinations thereof.

In another embodiment, the invention features a cell coupled to an imaging probe, where the imaging probe comprises a chromophore attachment moiety and one or more, e.g., a plurality of, chromophores wherein the chromophores are chemically linked to the chromophore attachment moiety so that upon activation of the imaging probe, a property of the chromophores are altered. The cell may be a transformed cell or a transformed cell that expresses the imaging probe.

A “chromophore” includes, but is not limited to, a fluorochrome, non-fluorochrome chromophore, fluorescence quencher, or absorption chromophore, including but not limited to organic and inorganic fluorochromes. Thus, in one embodiment, the imaging probe comprises a chromophore attachment moiety and a plurality of chromophores chemically linked to the chromophore attachment moiety so that upon activation, the optical properties of the chromophores are altered.

A “chromophore attachment moiety” is a biocompatible molecule, e.g., a backbone, to which two or more chromophores are chemically linked (directly or through a spacer) and maintained in spectral property altering permissive positions relative to one another. By “chemically linked” is meant connected by any attractive force between atoms strong enough to allow the combined aggregate to function as a unit. This includes, but is not limited to, chemical bonds such as covalent bonds (e.g., polar, or nonpolar), and non-covalent bonds such as ionic bonds, metallic bonds, and bridge bonds.

By “activation” of an imaging probe is meant any change to the probe that alters a detectable property, e.g., an optical property, of the probe. This includes, but is not limited to, any modification, alteration, or binding (covalent or non-covalent) of the probe that results in a detectable difference in properties, e.g., optical properties of the probe, e.g., changes in the fluorescence signal amplitude (e.g., dequenching and quenching), change in wavelength, fluorescence lifetime, spectral properties, or polarity. Optical properties include wavelengths, for example, in the visible, ultraviolet, near-infrared, and infrared regions of the electromagnetic spectrum. Activation can be, without limitation, by enzymatic cleavage, enzymatic conversion, phosphorylation or dephosphorylation, conformation change due to binding, enzyme-mediated splicing, enzyme-mediated transfer of the chromophore, hybridization of complementary DNA or RNA, analyte binding such as association with an analyte such as Na⁺, K⁺, Ca²⁺, Cl⁻, or another analyte, change in hydophobicity of the probe environment, and chemical modification of the chromophore. Activation of the optical properties may or may not be accompanied by alterations in other detectable properties, such as (but not limited to) magnetic relaxation and bioluminescence.

An “activation site” is a site which, upon activation, confers a detectable, e.g., conformational, change to the probe. For example, an activation site can be a covalent bond within a probe, wherein said bond is: (1) cleavable by an enzyme present in a target tissue, and (2) located so that its cleavage liberates a chromophore from being held in an optical-quenching interaction-permissive position.

“Optical-quenching interaction-permissive positions” are the positions of two or more atoms to which chromophores can be chemically linked (directly or indirectly through a spacer) so that the chromophores are maintained in a position relative to each other that permits them to interact photochemically and quench each other's emitted signal.

A “protective chain” is a biocompatible moiety covalently linked to the chromophore attachment moiety to inhibit undesired biodegradation, clearance, or immunogenicity of the probe.

A “targeting moiety” is a moiety bound covalently or noncovalently to a probe, which moiety enhances the concentration of the probe in a target tissue relative to surrounding tissue.

The invention also features an activatable imaging probe that is activated by phosphorylation or dephosphorylation of the probe. For example, the phosphorylation can be mediated by a kinase, and the dephosphorylation can be mediated by a phosphatase. The probes can have one or more phophorylation sites, and these sites can be, or be part of the chromophore attachment moiety, or can be within a spacer between the chromophore attachment moiety and the chromophores.

In another embodiment, the invention features an activatable imaging probe that includes a chromophore attachment moiety, a functional group, and one or more chromophores, wherein the chromophores are chemically linked to the chromophore attachment moiety so that upon activation of the probe the optical properties of the chromophores are altered, and wherein the probe is activated by enzyme-mediated removal of the functional group from the probe. The functional group can be chemically linked to the chromophore attachment moiety or to a spacer between the chromophore attachment moiety and the chromophores.

In another aspect, the invention also includes an activatable imaging probe that has a chromophore attachment moiety and one or more chromophores, wherein chromophores are chemically linked to the chromophore attachment moiety so that upon activation of the imaging probe the optical properties of chromophores are altered, and wherein the probe is activated by enzyme-mediated splicing. For example, the probe can include a nucleic acid sequence specific for enzyme-mediated splicing. The nucleic acid sequence specific for enzyme-mediated splicing can be, or be part of, the chromosome attachment moiety. Alternatively, the nucleic acid sequence can be within a spacer between the chromophore attachment moiety and the chromophores.

The new probes can also include a transmembrane signal sequence, e.g., one derived from a TAT protein comprising a caspase-3 sensitive cleavage site or one having the sequence Gly-Arg-Lys-Lys-Arg-Gln-Arg-Arg (SEQ ID NO:15) or Gly-Arg-Lys-Lys-Arg-Arg-Gln-Arg-Arg (SEQ ID NO:16).

The invention also features in vivo optical imaging methods. In one embodiment the methods include: (a) delivering to the subject an imaging probe of claim 1; (b) allowing adequate time for the imaging probe to be activated within the target; (c) illuminating the target with light of a wavelength absorbable by the chromophores; (d) detecting a signal emitted by the chromophores; and (e) forming an optical image from the emitted signal.

In these methods, steps (a)-(d) can be repeated at predetermined intervals to enable evaluation of the emitted signal of the chromophores in the subject over time. These methods can be used to detect a disease in the subject, or to characterize a phenotype or genotype and/or severity of a disease in the subject. The disease can be cancer, cardiovascular diseases, neurodegenerative diseases, immunologic diseases, autoimmune diseases, inherited diseases, infectious diseases, bone diseases, and environmental diseases.

The subject can be a mammal, including a human, or an animal model of a particular disease or disorder.

The invention also features an in vivo method for selectively imaging two or more cells or tissue types simultaneously. The method includes administering to a subject two or more activatable imaging probes, each of the two or more probes comprises a chromophore whose optical properties is distinguishable from that of the other chromophore, and each of the two or more probes contains a different activation site. The method therefore, allows the recording of multiple events. One or both of these probes (or different portions of the same probe) may be activatable or unchanged after target interaction, thereby providing local tissue concentration of probe delivery in addition to activation.

The methods of the invention can be used to determine a number of indicia, including tracking the localization of the imaging probe in a subject over time and assessing changes in the level of the imaging probe in the subject over time. The methods of the invention can also be used in the detection, characterization (i.e., genotype and phenotype) and/or determination of the localization of a disease, the severity of a disease or a disease-associated condition. Examples of such disease or disease-conditions include inflammation (e.g., inflammation that results in arthritis, for example, rheumatoid arthritis), all types of cancer, cardiovascular disease (e.g., atherosclerosis and inflammatory conditions of blood vessels), dermatologic disease (e.g., Kaposi's Sarcoma, psoriasis), ophthalmic disease (e.g., macular degeneration and diabetic retinopathy), infectious disease, immunologic disease (e.g., Acquired Immunodeficiency Syndrome, lymphoma, type I diabetes, and multiple sclerosis), neurodegenerative disease (e.g., Alzheimer's disease), and bone-related disease (e.g., osteoporosis and primary and metastatic bone tumors). The methods of the invention can therefore be used, for example, to determine the presence of tumor cells and localization of tumor cells, the presence and localization of inflammation, the presence and localization of vascular disease including areas at risk for acute occlusion (vulnerable plaques) in coronary and peripheral arteries and regions of expanding aneurysms, and the presence and localization of osteoporosis. The methods can also be used to follow therapy for such diseases by imaging molecular events modulated by such therapy, including but not limited to determining efficacy, optimal timing, optimal dosing levels (including for individual patients or test subjects), and synergistic effects of combinations of therapy.

A number of animal models are available and known in the art that mimic the progression and symptoms of several different human diseases. For example, animal models for multiple sclerosis, congestive heart failure, Alzheimer's disease, and Parkinson's disease have been established (Smith A H et al., 2000, J. Pharmacol. Toxicol. Methods, 43(2): 125; Hilliard, B et al., 2000, J. Immunol. 166(2): 1314; Yamada, K et al., 2000, Pharmacol. Ther. 88(2): 93; Bohn, M C et al., 2000, Novartis Found. Symp. 231(70), discussion 89-93). Moreover, with the advancements in recombinant technology, many new transgenic and gene knockout models are being developed (i.e., transgenic mice for breast cancer, Hutchinson, J N et al., 2000, Oncogene 19(53): 6130). These and other such models can be employed in the methods of the present invention.

The invention also features in vitro and in vivo optical imaging methods for assessing activity of an agent. In particular, the probes of the present invention may be used to assess molecular targets in vitro (e.g., in cell culture) and in vivo (e.g., animals or humans). The in vitro method for assessing the efficacy of an agent includes: (a) administering to the sample a new imaging probe; (b) allowing time for a molecule in the sample to activate the probe, if the molecule is present; (c) illuminating the sample with light of a wavelength absorbable by the chromophores; (d) detecting a signal emitted from the chromophores; (e) forming an optical image from the emitted signal; (f) administering to the sample the agent and repeating steps (a)-(e); and (g) comparing the emitted signals and images of steps (d) and (e) over time or at different agent doses to assess the activity of the agent. The sample can include, without limitation, cells, cell culture, tissue section, cytospin samples, or the like.

The in vivo method for assessing the efficacy of an agent includes: (a) administering to the subject an imaging probe; (b) allowing time for a molecule in a target tissue to activate the probe, if the molecule is present; (c) illuminating the target tissue with light of a wavelength absorbable by the chromophores; (d) detecting a signal emitted by the chromophores; (e) forming an optical image from the emitted signal; (f) administering to the subject the agent and repeating steps (a)-(e); and (g) comparing the emitted signals and images of steps (d) and (e) over time or at a different agent dose to assess activity of the agent. The subject may be a mammal, including a human.

In one embodiment, the methods are performed at least twice, once with and once without administering to the subject the agent, thereby providing a comparison of the outcome of the two methods for assessing the activity of the agent. The methods may also be performed prior to administration of the agent to determine whether a target (e.g., a drug target) is present and/or expressed, and therefore whether the agent should be administered to the subject. It is further appreciated that administration of the agent can be performed throughout the method including, without limitation, prior to administering the probe. It is also understood that a portion of the probe can be detected by other means (including second fluorescent wavelength, bioluminescence, changes in magnetic properties, or gamma radiation) or a second probe can be administered to determine the local concentration of the activatable probe, by any of the above means. The invention also includes a method for determining the presence of a composition (e.g., a drug or a polypeptide expressed by a gene, such as a gene introduced into the subject by gene therapy techniques) in a subject.

The agent can be any compound, including, but not limited to, therapeutic compounds. For example, the agent can be an enzyme inhibitor, e.g., a proteinase, kinase, transferase, or polymerase inhibitor, or their upstream regulators. The methods can therefore be used to identify the efficacy of therapeutic drug candidates. These methods can also be used to assess drug levels in a subject.

It will also be appreciated that the methods of the present invention may be used to optimize drug therapy, e.g., to optimize the dose, timing and/or administration route of a given therapeutic agent. The methods of the present invention may further be used for high throughput testing of therapeutic drug candidates (e.g., combinatorially designed therapeutic drug candidates). The methods can also be used to select drug candidates for clinical testing.

The invention also features in vivo optical imaging methods for guiding therapeutic, e.g., surgical, interventions by: (a) administering to a subject an imaging probe including a chromophore attachment moiety and a plurality of chromophores wherein the plurality of chromophores are chemically linked to the chromophore attachment moiety so that upon activation of the imaging probe, the optical properties of the chromophores are altered; (b) allowing time for molecules in a target tissue to activate the probe, if the molecules and/or target tissue are present; (d) illuminating the target tissue with light of a wavelength absorbable by the chromophores; and (e) detecting the optical signal emitted by the chromophores. The subject can be a mammal, including a human. The invention can be used to help a physician or surgeon to identify and characterize areas of disease, such as colon polyps or vulnerable plaque, to distinguish diseased and normal tissue, such as detecting tumor margins that are difficult to detect using an ordinary operating microscope. e.g., in brain surgery, and help dictate a therapeutic or surgical intervention, e.g., by determining whether a lesion is cancerous and should be removed or non-cancerous and left alone.

Unless otherwise defined, all technical and scientific terms used herein have the same meaning as commonly understood by one of ordinary skill in the art to which this invention belongs. Although methods and materials similar or equivalent to those described herein can be used in the practice or testing of the present invention, suitable methods and materials are described below. All publications, patent applications, patents, and other references mentioned herein are incorporated by reference in their entirety. In case of conflict, the present specification, including definitions, will control. In addition, the materials, methods, and examples are illustrative only and not intended to be limiting.

Other features and advantages of the invention will be apparent from the following detailed description, and from the claims.

### BRIEF DESCRIPTION OF THE DRAWINGS

FIGS. 1A and 1B are schematic diagrams indicating the chemical components, and their structural arrangement, in probes representing two embodiments of the invention.

FIGS. 2A and 2B are spectrophotometer scans of the near infrared chromophore, Cy5.5, before (FIG. 2A) and after (FIG. 2B) covalent linkage to PL-MPEG.

FIG. 3 is a bar graph summarizing data on intramolecular quenching and probe activation. The data were obtained using Cy-PL-MPEG probes with different levels of chromophore loading.

FIG. 4 is a schematic diagram illustrating the use of an endoscope in the invention.

### DETAILED DESCRIPTION

The invention features an imaging probe including a chromophore attachment moiety and one or more, e.g., a plurality of, chromophores wherein the chromophores are chemically linked to the chromophore attachment moiety so that upon activation of the imaging probe, the properties, e.g., optical properties, of the chromophores are altered. In one embodiment, the probe is intramolecularly quenched. In another embodiment, the imaging probe includes one or more quencher molecules that quench the initial signal, wherein dequenching of the chromophores occurs upon activation of the probe.

A chromophore attachment moiety can be any biocompatible backbone that allows a plurality of chromophores to be covalently linked thereto. In one embodiment, the chromophore attachment moiety is a polymer, for example, a polypeptide, a polysaccharide, a nucleic acid, or a synthetic polymer. Alternatively, the chromophore attachment moiety is a monomeric, dimeric, or oligomeric molecule. Polypeptides useful as the chromophore attachment moiety include, for example, polylysine, albumins, and antibodies. Poly(Llysine) is a useful polypeptide chromophore attachment moiety. The chromophore attachment moiety also can be a synthetic polymer such as polyglycolic acid, polylactic acid, polyglutamic acid, poly(glycolic-colactic) acid, polydioxanone, polyvalerolactone, poly-ε-caprolactone, poly(3-hydroxybutyrate, poly(3-hydroxyvalerate)polytartronic acid, and poly(β-malonic acid).

Activation sites can be located in the chromophore attachment moiety, e.g., when the chromophores are linked directly to ε-amino groups of polylysine. Alternatively, each chromophore can be linked to the chromophore attachment moiety by a spacer, e.g., a spacer containing a chromophore activation site. The spacers can be oligopeptides. Oligopeptide sequences useful as a spacer (or in a spacer) include: Arg-Arg; Arg-Arg-Gly; Gly-Pro-Ile-Cys-Phe-Phe-Arg-Leu-Gly (SEQ ID NO:1); His-Ser-Ser-Lys-Leu-Gln-Gly (SEQ ID NO:2); Gly-Arg-Lys-Lys-Arg-Arg-Gln-Arg-Arg-Arg-Lys(FITC)-Gly-Asp-Glu-Val-Asp-Gly-Cys(QSY7)-NH2 (SEQ ID NO:3); RRK(FITC)C-NH2 (SEQ ID NO: 4); GRRK(FITC)C-NH2 (SEQ ID NO:5); GRRRRK(FITC)C-NH2 (SEQ ID NO:6); GRRGRRK(FITC)C-NH2 (SEQ ID NO:7); GFGSVQ:FAGK(FITC)C-NH2 (SEQ ID NO:8); GFLGGK(FITC)C-NH2 (SEQ ID NO:9); Gly-Pro-Leu-Gly-Val-Arg-Gly-Lys(FITC)-Cys-NH2 (SEQ ID NO:10); Gly-D-Phe-Pip-Arg-Ser-Gly-Gly-Gly-Gly-Lys(FITC)-Cys-NH2 (Pip=pipecolic acid) (SEQ ID NO:11); and Gly-D-Phe-Pro-Arg-Ser-Gly-Gly-Gly-Gly-Lys(FITC)-Cys-NH2 (SEQ ID NO:12).

The imaging probe can include one or more protective chains covalently linked to the chromophore attachment moiety. Suitable protective chains include polyethylene glycol, methoxypolyethylene glycol, methoxypolypropylene glycol, copolymers of polyethylene glycol and methoxypolypropylene glycol, polylactic-polyglycolic acid, poloxamer, polysorbate 20, dextran and its derivatives, starch and starch derivatives, and fatty acids and their derivatives. In some embodiments of the invention, the chromophore attachment moiety is polylysine and the protective chains are methoxypolyethylene glycol.

Chromophores useful in the new probes include near infrared chromophores such as Cy5.5, Cy5, Cy7, IRD41, IRD700, NIR-1, IC5-OSu, LaJolla Blue, Alexaflour 660, Alexflour 680, FAR-Blue, FAR-Green One, FAR-Green Two, ADS 790-NS, ADS 821-NS, indocyanine green (ICG) and analogs thereof, indotricarbocyanine (ITC), chelated lanthanide compounds that display near infrared optical properties, and fluorescent quantum dots (zinc sulfide-capped cadmium selenide nanocrystals) (e.g., QuantumDot Corporation; www.qdots.com). The chromophores can be covalently linked to the chromophore attachment moiety including the spacers, using any suitable reactive group on the chromophore and a compatible functional group on the chromophore attachment moiety or spacer. A probe according to the present invention can also include a targeting moiety such as an antibody, antigen-binding antibody fragment, a receptor-binding polypeptide, a receptor-binding polysaccharide, or a hydrophobic region.

In another embodiment, the invention features a cell coupled to an imaging probe, where the imaging probe includes a chromophore attachment moiety and one or more, e.g., a plurality of, chromophores wherein the chromophores are chemically linked to the chromophore attachment moiety so that upon activation of the imaging probe, the optical properties of the chromophores are altered. The cell may be isolated from primary tissue, transformed, or genetically engineered to express the imaging probe. The imaging probe coupled to the cell may be used in the non-invasive in vivo optical imaging methods of the present invention.

The invention also features methods of optical imaging including the steps of delivering to a subject an imaging probe that includes a chromophore attachment moiety and a plurality of chromophores wherein the plurality of chromophores are chemically linked to the chromophore attachment moiety so that upon activation of the imaging probe, the optical properties of the chromophores are altered, allowing adequate time for the imaging probe to be activated within the target tissue, illuminating the target tissue with light of a wavelength absorbable by the chromophores, and detecting the signal emitted by the chromophores. These steps can be repeated at predetermined intervals thereby allowing the evaluation of emitted signal from the chromophore in a subject over time. The methods can be performed either in vivo or in vitro. The probe can also be coupled to a cell.

A cell coupled to an imaging probe is a cell expressing an imaging probe on its surface (e.g., an antibody or antibody fragment, a receptor or a ligand) or a cell transfected with a heterologous genetic construct that encodes an imaging probe. The cell can be prokaryotic or eukaryotic. Expression vectors containing a wide variety of regulatory elements are available and well known in the art. These vectors can be used to generate constructs capable of encoding an imaging probe. These constructs can be transiently transfected into a wide variety of cell types, including somatic cells, primary culture cells, and lymphoid cells. Alternatively, stable transfectants may be established from any number of well known cell lines, such as, but not limited to, HeLa, Daudi, K562, and COS cells.

Expression of the imaging probe in transfected cells can be regulated through the use of many different promoters known in the art. Constitutively active promoters such as CMV (cytomegalovirus) or SV40 (Simian Virus 40) can be used. Alternatively, inducible promoters such as the Tet system® and the Ecdysone-Inducible Expression System (with Ponasterone A)® (both available from Invitrogen, Inc.) can also be used and are commercially available and well known to those skilled in the art.

Probe Design and Synthesis

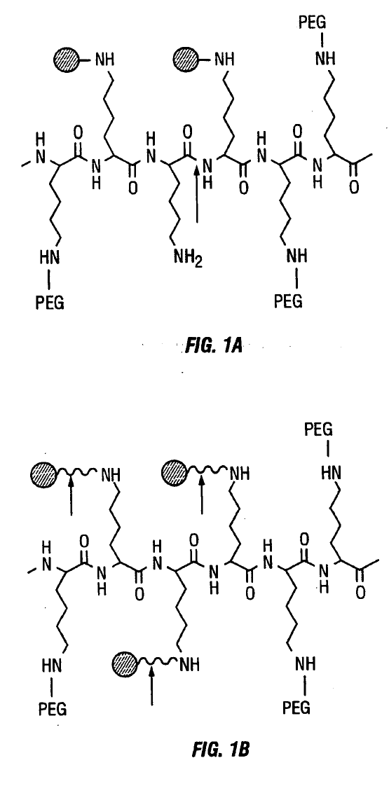

Probe architecture, i.e., the particular arrangement of probe components, can vary as long as the probe retains a chromophore attachment moiety, and optionally spacers, and one or more, e.g., a plurality of, chromophores, e.g., near infrared chromophores, linked to the chromophore attachment moiety so that upon activation of the imaging probe, the optical properties of the chromophores are altered. For example, the activation sites can be in the backbone itself, as shown in FIG. 1A, or in side chains, as shown in FIG. 1B. Although each chromophore in FIGS. 1A and 1B is in a separate side chain, a pair of chromophores can be in a single side chain. In such an embodiment, an activation site is placed in the side chain between the pair of chromophores.

In some embodiments, the probe comprises a polypeptide backbone containing only a small number of amino acids, e.g., 5 to 20 amino acids, with chromophores attached to amino acids on opposite sides of a protease cleavage (activation) site. Guidance concerning various probe components, including backbone, protective side chains, chromophores, chromophore attachment moieties, spacers, activation sites and targeting moieties is provided in the paragraphs below.

The chromophore attachment moiety design will depend on considerations such as biocompatibility (e.g., toxicity and immunogenicity), serum half-life, useful functional groups (for conjugating chromophores, spacers, and protective groups), and cost. Useful types of chromophore attachment moieties, also referred to herein as “backbones,” include polypeptides (polyamino acids), polyethyleneamines, polysaccharides, aminated polysaccharides, aminated oligosaccharides, polyamidoamines, polyacrylic acids, and polyalcohols. In some embodiments the backbone consists of a polypeptide formed from Lamino acids, D-amino acids, or a combination thereof. Such a polypeptide can be, e.g., a polypeptide identical or similar to a naturally occurring protein such as albumin, a homopolymer such as polylysine, or a copolymer such as a D-Tyr-D-Lys copolymer. When lysine residues are present in the backbone, the ε-amino “groups” on the side chains of the lysine residues can serve as convenient reactive groups for covalent linkage of chromophores and spacers (FIGS. 1A and 1B). When the backbone is a polypeptide, the molecular weight of the probe can be from 2 kD to 1000 kD, e.g., from 4 kD to 500 kD.

The chromophore attachment moieties can also be non-covalently associated complexes, such as liposomes. Chromophores may be attached to lipids before or after liposome formation. When these complexes interact with targets, the complexes can be activated, for example, without limitation, by quenching, de-quenching, wavelength shift, fluorescence energy transfer, fluorescence lifetime change, and polarity change. The probes can be located entirely within such a liposome and released locally with disruption of the liposome (such as with acoustic resonance energy imparted at ultrasound frequencies), or can be attached at the lipid surface.

A chromophore attachment moiety can be chosen or designed to have a suitably long in vivo persistence (half-life). Therefore, protective chains are not necessary in some embodiments of the invention. Alternatively, a rapidly biodegradable backbone such as polylysine can be used in combination with covalently linked protective chains. Examples of useful protective chains include polyethylene glycol (PEG), methoxypolyethylene glycol (MPEG), methoxypolypropylene glycol, polyethylene glycol-diacid, polyethylene glycol monoamine, MPEG monoamine, MPEG hydrazide, and MPEG imidazole. The protective chains can also be block-copolymers of PEG and a different polymer such as a polypeptide, polysaccharide, polyamidoamine, polyethyleneamine, or polynucleotide. Synthetic, biocompatible polymers are discussed generally in Holland et al., 1992, “Biodegradable Polymers,” Advances in Pharmaceutical Sciences, 6:101-164.

A useful backbone-protective chain combination is methoxypoly(ethylene)glycol-succinyl-N-ε-poly-L-lysyine (PL-MPEG). The synthesis of this material, and other polylysine backbones with protective chains, is described in Bogdanov et al., U.S. Pat. No. 5,593,658 and Bogdanov et al., 1995, Advanced Drug Delivery Reviews, 16:335-348.

Modifications to the chromophore attachment moiety can also be made to improve delivery and activation. For example, graft copolymers can be modified to improve both the probes' biological properties and/or improve activation. For example, a 560 kD MPEG-PL graft copolymer randomly modified with Cy5.5 to yield a cathepsin B-sensitive probe (as described in the examples of U.S. Pat. No. 6,083,486) was further modified to yield a succinilated probe, i.e., the positive charges on the probe were modified to neutral or negative charges by acetylation or succinilation, respectively, which demonstrated improved activation properties.

There are numerous other chemical modifications of polymers that can be made, including changes in the charge of the polymer, changes in the polymers' hydrophobic and hydrophilic properties, changes in the size and length of the polymer side chains, and addition of attractants and/or binding moieties for enzymes. Examples of such modifications include a large number of small molecules such as succinate, acetate, amino acids phenyl, guanidinium, tetramethylguanidinium, methyl, ethyl, propyl, isopropyl, and benzyl.

Membrane translocation signals can also be added to the imaging probes to improve deliverability. Since many graft copolymers can enter various cell types through fluid phase endocytosis, improvement of cellular uptake and assurance of cytoplasmic deposition of the imaging probe can be achieved by attaching membrane translocation (or transmembrane) signal sequences. These signal sequences can be derived from a number of sources including, without limitation, viruses and bacteria. For example, a Tat protein-derived peptide containing a caspase-3 sensitive cleavage site with the sequence—Gly-Arg-Lys-Lys-Arg-Arg-Gln-Arg-Arg-Arg-Lys(FITC)-Gly-Asp-Glu-Val-Asp-Gly-Cys(QSY7)-NH2—(SEQ ID NO:3) has been shown to be efficiently internalized into cells for monitoring caspase-3 activity. The sequences Gly-Arg-Lys-Lys-Arg-Gln-Arg-Arg (SEQ ID NO:15) or Gly-Arg-Lys-Lys-Arg-Arg-Gln-Arg-Arg (SEQ ID NO:16) can also be used.

Other targeting and delivery approaches can also be used such as folate-mediated targeting (Leamon & Low, 2001, Drug Discovery Today, 6:44-51), liposomes, transferrin, vitamins, carbohydrates and the use of other ligands that target internalizing receptors, including, but not limited to, somatostatin, nerve growth factor, oxytocin, bombesin, calcitonin, arginine vasopressin, angiotensin II, atrial nati-uretic peptide, insulin, glucagons, prolactin, gonadotropin, and various opioids. In addition, other ligands can be used that upon intracellular delivery, undergo an enzymatic conversion that leaves the resulting conversion product trapped within the cell, such as nitroheteroaromatic compounds that are irreversibly oxidized by hypoxic cells.

Various near infrared chromophores are commercially available and can be used to construct probes according to this invention. Exemplary chromophores include the following: Cy5.5, Cy5 and Cy7 (Amersham, Arlington Hts., IL); IRD41 and IRD700 (LI-COR, Lincoln, Nebr.); NIR-1 and IC5-OSu, (Dejindo, Kumamoto, Japan); Alexflour 660, Alexflour 680 (Molecular Probes, Eugene, Oreg.), LaJolla Blue (Diatron, Miami, Fla.); FAR-Blue, FAR-Green One, and FAR-Green Two (Innosense, Giacosa, Italy), ADS 790-NS and ADS 821-NS (American Dye Source, Montreal, Canada), indocyanine green (ICG) and its analogs (Licha et al., 1996, SPIE 2927:192-198; Ito et al., U.S. Pat. No. 5,968,479); indotricarbocyanine (ITC; WO 98/47538); fluorescent quantum dots (zinc sulfide-capped cadmium selenide nanocrystals) (QuantumDot Corporation; www.qdots.com) and chelated lanthanide compounds. Fluorescent lanthanide metals include europium and terbium. Fluorescence properties of lanthanides are described in Lackowicz, 1999, Principles of Fluorescence Spectroscopy, 2^{nd} Ed., Kluwar Academic, New York.

Imaging probes with excitation and emission wavelengths in the near infrared spectrum are preferred, i.e., 650-1300 nm. Use of this portion of the electromagnetic spectrum maximizes tissue penetration and minimizes absorption by physiologically abundant absorbers such as hemoglobin (<650 nm) and water (>1200 nm). Ideal near infrared chromophores for in vivo use exhibit the following characteristics: (1) narrow spectral characteristics, (2) high sensitivity (quantum yield), (3) biocompatibility, and (4) decoupled absorption and excitation spectra. Table 1 summarizes information on the properties of six commercially available near infrared chromophores.

TABLE 1
Exemplary Near Infrared Chromophores

| | λ(nm) | λ(nm) | Mol. | Extinct. | Quantum |
|---|---|---|---|---|---|
| Fluorochrome | excitation | emission | Wt. | Coef. | yield % |
| Cy5.5 | 675 | 694 | 1128.41 | 250,000 | 28.0 |
| Cy5 | 649 | 670 | 791.99 | 250,000 | 28.0 |
| Cy7 | 743 | 767 | 818.02 | 200,000 | 28.0 |
| IRD41 | 787 | 807 | 925.10 | 200,000 | 16.5 |
| IRD700 | 685 | 705 | 704.92 | 170,000 | 50.0 |
| IC5-OSu | 641 | 657 | 630.3 | NA | NA |
| NIR-1 | 663 | 685 | 567.08 | 75,000 | NA |
| LaJolla Blue | 680 | 700 | 5000.00 | 170,000 | 70.0 |
| Alexa Fluor 660 | 663 | 690 | 1100 | 132,000 | NA |
| Alexa Fluor 680 | 679 | 702 | 1150 | 184,000 | NA |
| ADS 790 NS | 791 | >791 | 824.07 | NA | NA |
| ADS 821 NS | 820 | >820 | 924.07 | NA | NA |
| Far-Blue | 660 | 678 | 825 | 150,000 | NA |
| Far-Green One | 800 | 820 | 992 | 150,000 | NA |
| Far-Green Two | 772 | 778 | | 150,000 | NA |
| ICG | 780 | 812 | 774.98 | 115,000 | 1.2 |
| ITC* | 753 | 790 | 1089 | 201000 | 6.6 |

*See WO 98/47538

Although near infrared chromophores can be used, it will be appreciated that the use of chromophores with excitation and emission wavelengths in other spectrums, such as the visible light spectrum, can also be employed in the compositions and methods of the present invention.

Intramolecular quenching by non-activated probes can occur by any of various quenching mechanisms. Several mechanisms are known including resonance energy transfer between two chromophores. In this mechanism, the emission spectrum of a first chromophore should be very similar to the excitation of a second chromophore, which is in close proximity to the first chromophore. Efficiency of energy transfer is inversely proportional to r⁶, where r is the distance between the quenched chromophore and excited chromophore. Self-quenching can also result from chromophore aggregation or excimer formation. This effect is concentration dependent. Quenching also can result from a nonpolar-to-polar environmental change.

To achieve intramolecular quenching, several strategies can be applied. They include: (1) linking a second chromophore, as an energy acceptor, at a suitable distance from the first chromophore; (2) linking chromophores to the backbone at high density, to induce self-quenching; and (3) linking polar chromophores in a vicinity of non-polar structural elements of the backbone and/or protective chains. Partial or full recovery of the optical properties can be protected upon cleavage of the chromophore from neighboring chromophores and/or from a particular region, e.g., a non-polar region, of the probe.

The chromophore can be covalently linked to a chromophore attachment moiety or spacer using any suitable reactive group on the chromophore and a compatible functional group on the chromophore attachment moiety or spacer. For example, a carboxyl group (or activated ester) on a chromophore can be used to form an amide linkage with a primary amine such as the ε-amino group of the lysyl side chain on polylysine.

In some embodiments of the invention, chromophores are linked to the chromophore attachment moiety through spacers containing activation sites. For example, oligopeptide spacers can be designed to contain amino acid sequences recognized by specific proteases associated with target tissues. Some probes of this type accumulate in tumor interstitium and inside tumor cells, e.g., by fluid phase endocytosis. By virtue of this accumulation, such probes can be used to image tumor tissues, even if the enzyme(s) activating the probe are not tumor specific.

In other embodiments of the invention, two paired chromophores in quenching positions are in a single polypeptide side chain containing an activation site between the two chromophores. Such a side chain can be synthesized as an activatable module that can be used as a probe per se, or linked to a backbone or targeting moiety, e.g.; an albumin, antibody, receptor binding molecule, synthetic polymer or polysaccharide. A useful conjugation strategy is to place a cysteine residue at the N-terminus or C-terminus of the molecule and then employ SPDP for covalent linkage between the side chain of the terminal cysteine residue and a free amino group of the carrier or targeting molecule.

In other embodiments, various enzymes activate the new probes by cleavage. For example, Prostate Specific Antigen (PSA), is a 33 kD chymotrypsin-like serine protease secreted exclusively by prostatic epithelial cells. Normally, this enzyme is primarily involved in post-ejaculation degradation of the major human seminal protein, and PSA concentrations are proportional to the volume of prostatic epithelium. The release of PSA from prostate tumor cells, however, is about 30-fold higher than that from normal prostate epithelium cells. Damage to basal membrane and deranged tissue architecture allow PSA to be secreted directly into the extracellular space and into the blood. Although high levels of PSA can be detected in serum, the serum PSA exists as a complex with a1-antichymotrypsin protein, and is proteolytically inactive. Free, uncomplexed, activated PSA is present in the extracellular fluid from malignant prostate tissues, and PSA activity can be used as a marker for prostate tumor tissue. Moreover, prostate tumor tissue is highly enriched in PSA, therefore, spacers containing the amino acid sequence recognized by PSA can be used to produce an imaging probe that undergoes activation specifically in prostate tumor tissue. An example of a PSA-sensitive spacer is His-Ser-Ser-Lys-Leu-Gln-Gly (SEQ ID NO:2). Other PSA-sensitive spacers can be designed using information known in the art regarding the substrate specificity of PSA. See, e.g., 1997, Denmeade et al., Cancer Res. 57:4924-4930.

Another example involves Cathepsin D, an abundant lysosomal aspartic protease distributed in various mammalian tissues. In most breast cancer tumors, cathepsin D is found at levels from 2-fold to 50-fold greater than levels found in fibroblasts or normal mammary gland cells. Thus, cathepsin D can be a useful marker for breast cancer. Spacers containing the amino acid sequence recognized by cathepsin D can be used to produce an imaging probe that undergoes activation specifically in breast cancer tissue. An example of a cathepsin D-sensitive spacer is the oligopeptide: Gly-Pro-Ile-Cys-Phe-Phe-Arg-Leu-Gly (SEQ ID NO:1). Other cathepsin D-sensitive spacers can be designed using information known in the art regarding the substrate specificity of cathepsin D. See, e.g., Gulnik et al., 1997, FEBS Let., 413:379-384.

Another example involves matrix metalloproteinases (MMPs). Several MMPs are expressed in cancers at much higher levels than in normal tissue and the extent of expression has been shown to be related to tumor stage, invasiveness, metastasis, and angiogenesis. MMP-2 (gelatinase) in particular, has been identified as one of the key MMPs in these processes, being capable of degrading type IV collagen, the major component of basement membranes. Based on these observations, several companies have initiated the development of different MMP inhibitors to treat malignancies and other diseases involving pathologic angiogenesis.

The design of proteinase inhibitors has evolved over the last decade and now largely relies on structure-based designs, screening of combinatorial libraries, or employing other combinatorial peptide approaches. Through these efforts, a number of broad-spectrum and more “selective” MMP inhibitors have been described and are in clinical trials, while a number of agents are in preclinical development. Efficacy testing in animals has largely been measured as suppression of tumor growth based on tumor volume measurement following treatment and by assessment of histological and anti-angiogenic effects of MMP inhibitors in human tumor xenografts. However, differences in tumor growth usually do not reach statistical significance in murine models until 10-20 days after initiation of treatment. In a clinical setting, surrogate markers of treatment efficacy such as tumor regression, time to recurrence or time to progression have been used because of the lack of more direct measures, although the limitations of such late endpoints are obvious.

MMP inhibitors may also be more effective when used in combination with chemotherapeutic agents. A specific molecular target-based pharmacodynamic assessment of each therapeutic approach would therefore be highly desirable (for estimating the relative contributions of each agent and resulting synergies). For the reasons outlined above there is a need to directly detect and monitor proteinase activities in vivo in an intact tumor environment.

Spacers containing the amino acid sequence recognized by MMP-2 can be used to produce an imaging probe that undergoes activation specifically in cancer tissue expressing MMP-2. An example of a MMP-2-sensitive spacer is the oligopeptide: GPLGVRGK(FITC)C-NH₂ (SEQ ID NO:10). Other MMP-2-sensitive spacers can be designed using information known in the art regarding the substrate specificity of MMP-2. In addition, other MMP probes can be designed accordingly.

Various other enzymes can be exploited to provide probe activation (cleavage) in particular target tissues in particular diseases. Table 2 provides information on several exemplary enzymes and associated diseases (See Barrett et al. Handbook of Proteolytic Enzymes, 1998 Academic Press).

TABLE 2
Enzyme-Disease Associations

| Enzyme | Disease | Reference |
|---|---|---|
| Cathepsin B | Cancer, Cardiovascular | Nat. Biotech., 1999; 17: 375 |
| | Disease, Arthritis, | |
| | Neurodegenerative disease | |
| Cathepsin D | Cancer | Gulnik, 1997, FEBS Lett., |
| | | 413: 379. |
| Cathepsin K | Osteoporosis | Bone, 2000, 26: 241-247. |
| | Bone Cancer | |
| Cathepsin X | Cancer | Biochemistry, 1999, |
| | | 38: 12648-54. |
| Cathepsin S | Allergy, Asthma | J. Clin. Invest., 1998 |
| | | 101: 2351-63. |
| Caspases | Apoptosis, Ischemia, | P.N.A.S., 1996: |
| | Arthritis, | 93: 14559-63 |
| | Neurodegenerative disease, | |
| | Cardiovascular Disease | |
| PSA | Prostate Cancer | Denmeade, 1997, Cancer |
| | | Res. 57: 4924. |
| MMP's | Cancer, Metastases, | Verheijen, 1997, Biochem. J. |
| | Inflammation, Arthritis, | 323: 603. |
| | Multiple Sclerosis, Macular | |
| | degeneration, | |
| | Cardiovascular Disease | |
| CMV | Viral | Sardana, 1994, J. Biol. |
| protease | | Chem. 269: 14337 |
| Thrombin | Blood clotting | Rijkers, 1995, Thrombosis |
| | | Res., 79: 491. |
| Beta- | Alzheimer Disease | J. Biol. Chem., 2001. In |
| secretase | | Press |
| (BACE) | | |
| Urokinase | Cancer | Clin. Cancer Res., 2001, |
| plasminogen | | 7: 2396. |
| activator | | |

Protease cleavage sites can be determined and designed using information and techniques known in the art including using various compound and peptide libraries and associated screening techniques (Turk et al., 2001, Nature Biotech., 19:661-667).

In one embodiment of the present invention, when the chromophores are linked directly to the backbone, probe activation may be by cleavage of the backbone. High chromophore loading of the backbone can interfere with backbone cleavage by activating enzymes such as cathepsins. Therefore, a balance between signal quenching and accessibility of the backbone by probe-activating enzymes is important. For any given backbone-chromophore combination (when activation sites are in the backbone) probes representing a range of chromophore loading densities can be produced and tested in vitro to determine the optimal chromophore loading percentage.

When the chromophores are linked to the backbone through activation site-containing spacers, accessibility of the backbone by probe-activating moieties is unnecessary. Therefore, high loading of the backbone with spacers and chromophores does not significantly interfere with probe activation. For example, in such a system, every lysine residue of polylysine can carry a spacer and chromophore, and every chromophore can be released by activating enzymes.

Accumulation of a probe in a target tissue can be achieved or enhanced by binding a tissue-specific targeting moiety to the probe. The binding can be covalent or non-covalent. Examples of targeting moieties include a monoclonal antibody (or antigen-binding antibody fragment) directed against a target-specific marker, a receptor-binding polypeptide directed to a target-specific receptor, and a receptor-binding polysaccharide directed against a target-specific receptor.

Antibodies or antibody fragments can be produced and conjugated to probes of this invention using conventional antibody technology (see, e.g., Folli et al., 1994, “Antibody-Indocyanin Conjugates for Immunophotodetection of Human Squamous Cell Carcinoma in Nude Mice,” Cancer Res., 54:2643-2649; Neri et al., 1997, “Targeting By Affinity-Matured Recombinant Antibody Fragments of an Angiogenesis Associated Fibronectin Isoform,” Nature Biotechnology, 15:1271-1275). Similarly, receptor-binding polypeptides, such as somatostatin peptide, and receptor-binding polysaccharides can be produced and conjugated to probes of this invention using known techniques. Other targeting and delivery approaches can also be used such as folate-mediated targeting approaches (Leamon & Low, 2001, Drug Discovery Today, 6:44-51), liposomes, transferrin, vitamins, carbohydrates and use of other ligands that target internalizing receptors including but not limited to nerve growth factor, oxytocin, bombesin, calcitonin, arginine vasopressin, angiotensin II, atrial nati-uretic peptide, insulin, glucagons, prolactin, gonadotropin, and various opioids. In addition, other ligands can be used that upon intracellular delivery, undergo an enzymatic conversion that leaves the resulting conversion product trapped in the cell, such as nitroheteroaromatic compounds that are irreversibly oxidized by hypoxic cells.

In one embodiment, activation of the imaging probe can be achieved through phosphorylation or dephosphorylation of the probe. Phosphorylation is mediated through enzymes such as kinases, which are abundantly involved in signal transduction and function by adding a phosphate group to either serine, threonine or tyrosine amino acids. There are a number of different types of kinases including, without limitation, receptor tyrosine kinases, the Src family of tyrosine kinases, serine/threonine kinases and the Mitogen-Activated Protein (MAP) kinases. In addition, many of these molecules are associated with various disease states. Examples of kinases useful in the present invention and their associated diseases are listed in Table 3.

TABLE 3
Kinase - Disease Associations

| Kinase Type | Examples | Associated Diseases |
|---|---|---|
| Receptor Tyrosine Kinases | 1. Epidermal Growth Factor | 1. cancers of the digestive tract, |
| | Receptor (EGFR) | breast and colorectal cancer |
| | 2. Her2/neu | 2. breast cancer |
| | 3. Platelet-Derived Growth | 3. fibroadenomas of the breast |
| | Factor (PDGF) | |
| | 4. Vascular Endothelial Growth | 4. angiogenesis |
| | Factor (VEGF) | |
| | 5. Insulin receptor | 5. diabetes mellitus |
| Src family | 1. Lyn | 1. Wiskott-Aldrich syndrome |
| | 2. Fyn | 2. Wiskott-Aldrich syndrome |
| | 3. Bruton's Tyrosine Kinase | 3. X-Linked ammaglobulinemia |
| | (BTK) | |
| Serine/Threonine | 1. Protein Kinase C (PKC) | 1. Diabetes-mellitus-related |
| | 2. cardiovascular complications | 2. Alzheimer's syndrome |
| Mitogen-Activated Protein (MAP) | p38 | Inflammation |
| kinases | | |

Thus, in one embodiment of the present invention, phosphorylation is used to activate the probe. The phosphorylation of the serine, threonine, or tyrosine amino acids will cause attraction of the negatively charged phosphate groups to the positively charged groups on the opposite molecule, thus bringing the chromophores into an interactive permissive position, causing changes in their optical parameters, e.g., quenching, dequenching, wavelength shift, fluorescence energy transfer, fluorescence life time change, or polarity change. The molecules can be fluorescence dyes, quenchers, and/or inducers (i.e., a compound which causes fluorescence lifetime change or polarity change). Phosphorylation may also increase the local hydrophilicity, thus decreasing the fluorescent resonance energy transfer between fluorochromes that is dependent upon local solvent concentration (e.g., resulting in decreased quenching).

In another embodiment, activation can be accomplished by utilizing an enzyme that removes or modifies a functional group (e.g., a phosphate group) located on the spacer of the probe. The probe is thus modified to incorporate a target sequence or chemical structure into a spacer that is then modified or removed from the spacer in order to activate the probe. In one example, a phosphate-ester metabolizing enzyme such as an alkaline or acid phosphatase is used. These enzymes hydrolyze phosphate monoesters to an alcohol and inorganic phosphate. Examples of enzymes useful in the present invention include conjugates of calf intestinal alkaline (CIP) phosphatase and PTPIB and PTEN phosphatase inhibitors, both of which have been currently developed for diabetes and gliomas, respectively.

In another embodiment of the present invention, other forms of chemical modification can be utilized to activate the probe, such as methylation. Methylase enzymes covalently link methyl groups to adenine or cysteine nucleotides within restriction enzyme target sequences, thus rendering them resistant to cleavage by restriction enzymes. A methylation enzyme such as S-adenosylmethionine may therefore be used to methylate a spacer of the imaging probe, thus rendering a quencher molecule resistant to restriction enzyme cleavage. Alternatively, a demethylase such as purified 5-MeC-DNA glycosylase may be used to demethylate a spacer, thus allowing restriction enzyme cleavage of a quenching molecule and the subsequent dequenching of the chromophore.

In another embodiment of the present invention, probes containing mismatches or mutations in their sequence are provided wherein the function of specific DNA repair enzymes is used to activate the probe. For example, a mismatch within the spacer of the imaging probe, results in the signal being quenched. Upon the correction of this mismatch by the appropriate DNA enzyme, a conformational change occurs allowing the dequenching of the signal. There are several enzymes involved in DNA repair, including, without limitation, poly ADP-ribose polymerase (PARP), DNA polymerases α, β, and Σ and DNA ligase. Several human diseases are a result of deficiencies in DNA repair, including Ataxia-Telangiectasia, Xeroderma Pigmentosum, Cockayne Syndrome, and Santis-Caccione Syndrome. The loss of mismatch repair enzyme function has also been associated with the early development of many cancers.

Mutations can be inserted into the probe DNA in several different ways. For example, some methods of mutagenesis include: (1) utilizing degenerate oligonucleotides to create numerous mutations in a small DNA sequence; (2) spacer-scanning using nested deletions and complementary nucleotides to insert point mutations throughout a sequence of interest; (3) spacer-scanning using oligonucleotide-directed mutagenesis; and (4) utilizing the polymerase chain reaction (PCR) to generate specific point mutations.

In another embodiment, ubiquitin-specific target sequences can be added to the probe wherein the ubiquination of the target sequence allows for the chromophores to be brought into close proximity, permitting energy transfer between the chromophores, thus activating the probe through any of thee mechanisms listed herein. Ubiquination is an important process in the regulation of many biological processes, including angiogenesis and oxygen sensing. For example, the product of the von Hippel-Lindau (VHL) tumor suppressor gene (pVHL), whose loss of function contributes to VHL disease and also contributes to 70% of renal cell carcinomas, has been shown to directly promote degradation of Hypoxia-Indicuble-Factor (HIF) by ubiquination (Cockman et al., J., Biol. Chem., 2000, 275:25733-25741; Ohh et al., Nature Cell Biol., 2000, 2:423-427). Inhibitors of the ubiquination pathway include Lactocystin and the Calpain I inhibitor LLnL (N-acetyl-Leu-Leu-Norleucinal) (J. Biomol. Screen, 2000, 5(5): 319-328).

In another embodiment of the present invention, specific target binding sites can be incorporated into the probe. These can include, without limitation, peptide substrates, enzyme binding sites, peptide sequences, sugars, RNA or DNA sequences, or other specific target binding sites or moieties. The probe is activated upon the binding of the target binding site, e.g., a change in the spectral properties of the chromophore occurs, for example, by adequate separation between the spacer and quencher. This is commonly referred to as a “molecular beacon.” Tyagi, 1998, Nature Biotech., 16:49.

A number of specific peptide substrates including cathepsin B-specific peptide substrates, MMP substrates, thrombin substrates and others are included in the probes of the present invention (see, e.g., Table 2). Examples of cathepsin B-specific substrates include RRK(FITC)C-NH₂ (SEQ ID NO:4), GRRK(FITC)C-NH₂ (SEQ ID NO:5), GRRRRK(FITC)C-NH₂ (SEQ ID NO:6), GRRGRRK(FITC)C-NH₂ (SEQ ID NO:7), GFGSVQ:FAGK(FITC)C-NH₂ (SEQ ID NO:8) (Bioconjugate Chem 1999, 553), and GFLGGK(FITC)C-NH₂ (SEQ ID NO:9), (Bioconjugate Chem 2000, 132). An example of a MMP substrate is Gly-Pro-Leu-Gly-Val-Arg-Gly-Lys(FITC)-Cys-NH₂ (SEQ ID NO:10). Examples of thrombin-specific substrates (Rijkers D., Thrombosis Research 1995, 79, 491) include Gly-D-Phe-Pip-Arg-Ser-Gly-Gly-Gly-Gly-Lys(FITC)-Cys-NH₂ (Pip=pipecolic acid) (SEQ ID NO:11), Gly-D-Phe-Pro-Arg-Ser-Gly-Gly-Gly-Gly-Lys(FITC)-Cys-NH₂ (SEQ ID NO:12).

A monoclonal antibody (or antigen-binding antibody fragment) directed against a target-specific marker or a receptor-binding polypeptide or polysaccharide directed against a target-specific receptor may also be used to activate the probe. Specific proteins include, but are not limited to, G protein coupled receptors, nuclear hormone receptors such as estrogen receptors, and receptor tyrosine kinases.

In another embodiment of the present invention, enzymes that are capable of transferring the chromophore are used to activate the probe. Specific target sequences that are recognized by enzymes involved in recombination of DNA (recombinases) are incorporated into the probe. Upon recognition of the target site by the enzyme, the chromophore is transferred to another molecule (recombination) resulting in altered spectral properties of the chromophore or removal or alteration of the quencher from the spacer. Enzymes involved in recombination are well known in the art. For example, recombinases are involved in immunoglobulin (Ig) and T cell receptor (TCR) gene rearrangements, a process involving the recombination of non-homologous gene segments, which occurs in immature B and T cells. The genes that encode these recombinases have been cloned and identified as RAG-1 and RAG-2.

In another embodiment, the probes can be activated by incorporating into the probe target sequences for enzymes involved in RNA splicing. This embodiment involves incorporating an RNA splicing sequence (e.g., an intron segment) on the spacer portion of the probe, resulting in the alteration of the spacer length. Activation is accomplished by either changing the spectral properties of the chromophore or by the removal or alteration of the quencher from the spacer of the probe. Several methods of RNA splicing are known in the art. For example, splicing of introns from mRNA is mediated by a group of enzymes known as small nuclear RNAs (snRNAs) which complex together to form a splicosome. These enzymes splice RNA by precisely breaking sugar-phosphate bonds at the boundaries of introns and rejoining the free ends generated by intron removal into a continuous mRNA molecule. There are also alternative splicing pathways that allow for the formation of several different but related mRNAs that in turn encode for different but related proteins. For example, the thyroid hormone calcitonin and the calcitonin gene-related polypeptide found in hypothalamus cells are derived from the same pre-mRNA species, but due to alternative splicing, result in two different, but related proteins.

In another aspect, the invention features a fluorescent probe including a fluorochrome attachment moiety and a plurality of fluorochromes wherein the plurality of fluorochromes are chemically linked to the fluorochrome attachment moiety so that upon “activation” of the fluorescent probe by an analyte, the spectral properties of the fluorochromes are altered.

An “analyte” refers to a molecule or ion that binds to and activates fluorescent probes. Such analytes include, but are not limited to H⁺, Ca²⁺, Na⁺, Mg²⁺, Mn²⁺, Cl⁻, Zn²⁺, O₂, NO, Fe²⁺, K⁺, and H₂O₂.

In one embodiment of the invention, analyte binding is used to activate the probe. The binding of the analyte to the activation site causes an analyte-induced conformational change, thus bringing the fluorochromes into an interaction permissive position, causing changes in their optical parameters, e.g., quenching, dequenching, wavelength shift, fluorescence energy transfer, fluorescence life time change, or polarity change. The molecules can be fluorescent dyes, quenchers, and/or inducers (i.e., a compound which causes a fluorescence lifetime change or polarity change).

Peptides and polypeptides that selectively bind to analytes and undergo analyte-induced conformational changes are known, including peptides based on zinc finger domains and calcium binding EF-hand domains (See, e.g., Berg and Merckle, J. Am. Chem. Soc., 1989, 111:3759-3761; Krizek et al., Inorg. Chem., 1993, 32:937-940; Krizek and Berg, Inorg. Chem., 1992, 31:2984-2986; Kim et al., J. Biol. Inorg. Chem., 2001, 6:173-81; and U.S. Pat. No. 6,197,928). A single zinc finger domain is 25-30 amino acids in length and has the consensus sequence (F/Y)-X-C-X₂₋₄-C-X₃-F-X₅-L-X₂-H-X₃₋₅-H-X₂₋₆ (SEQ ID NO:13), where X is any amino acid (Berg, Acc. Chem. Res., 1995, 28:14-19).

A single EF-domain is a helix-loop-helix motif that usually has 12 residues with the pattern, X-A-X-A-X-A-X-A-X-A-A-X (SEQ ID NO:14), where X is an amino acid that participates in metal coordination, e.g., histidine, glutamic acid, or aspartic acid, and A represents the intervening amino acids, which can be any amino acid (Bently, A. L. and Rety, S., Curr. Opin. Struct. Biol., 2000, 10:637-643).

Other peptide sequences and methods to design and screen for peptides that bind to specific analytes are also known (Bar-Or, et al., Eur. J. Biochem., 2001, 268:42-47; Enzelberger et al., J. Chromatogr. A., 2000, 10:83-94; Fattorusso, et al., Biopolyers, 1995, 37:401-410; Bonomo et al., Chemistry, 2000, 6:4195-4202; Ashraf et al., Bioorg. Med. Chem., 2000, 10:1617-1620; Zoroddu ey al., J. Inorg. Biochem., 2001, 84:47-54; Mukhejee and Chattopadhyay, Indian Chem. Soc., 1991, 68:639-642; Hulsbergen and Reedijk, Recl. Trav. Chim. Pays-Bas, 1993, 112:278-286; Ama et al., Bull Chem. Soc. Japan, 1989, 62:3464-3468; U.S. Pat. No. 6,083,758, Method for Screening Peptides for Metal Coordinating Properties and Fluorescent Chemosensors Derived Therefrom; and U.S. Pat. No. 5,928,955, Peptidyl fluorescent Chemosensors for Divalent Zinc).

In another embodiment of the invention, probe activation can be achieved by using the fluorochrome itself as a molecule that changes spectral properties after interaction with and/or binding to a specific analyte. Many fluorochrome molecules that exhibit altered spectral properties after interaction with a specific analyte are commercially available and are well known (See Tsien R. Y., 1992, Probe of dynamic biochemical signals inside living cells. In Fluorescent Chemosensors for Ion and Molecular Recognition, edited by Czarnik, A. W. pg. 130-146. ACS Books, Washington, D.C.; Tsien, R. Y., Biochemistry, 1980, 19:2396-2404; Grynkiewicz et al., J. Biol. Chem., 1985, 260:3440-3450; www.molecularprobes.com; www.biotium.com; U.S. Pat. No. 5,134,232, Fluorescent indicator dyes for alkali metal cations; and U.S. Pat. No. 5,393,514, Fluorescent pH indicators).

Examples of several commercially available fluorochrome sensors/indicators molecules are listed in Table 4. Several of these fluorochrome molecules are commercially available as succimidyl esters that can be easily conjugated to primary amine groups, e.g., of peptides or other biologically compatible molecules. Although near-infrared fluorochromes are useful, it will be appreciated that the use of fluorochromes with excitation and emission wavelengths in other spectrums, such as the visible light spectrum, can also be employed in the compositions and methods of the present invention.

TABLE 4

| | λ(nm) | λ(nm) | | |
|---|---|---|---|---|
| Fluorochrome | excitation | emission | Analyte | Best Detection Mode |
| DHPN | 360/420 | 455/512 | H⁺ | Emission Ratio |
| BCECF | 440/490 | 530 | H⁺ | Excitation Ratio |
| SNARF-1 | 517/576 | 587/640 | H⁺ | Emission Ratio |
| PBFI | 340/350 | 530 | K⁺ | Excitation Ratio |
| | | | | or Intensity |
| SBFI | 340/385 | 530 | Na⁺ | Excitation Ratio |
| Fluo-3 | 500 | 530 | Ca²⁺ | Intensity |
| Rhod-2 | 522 | 581 | Ca²⁺ | Intensity |
| OxyPhor-R2 | 419/524 | | O₂ | Lifetime measurement |

Many of these molecules, and others like them, have been used in vivo. For example. BCECF has been used in vivo to measure the pH of gastrointestinal mucosa, which is an important factor in the detection of hypoxia-induced dysfunctions (Marechal et al., Photochem. Photobiol., 1999, 70:813-819) as well as for intracellular pH measurement during cerebral ischemia and reperfusion (Itoh et al., Keio J. Med., 1998, 47:37-41) and for non-invasively monitoring the in vivo pH in conscious mice (Russell et al., Photochem. Photobiol., 1994, 59:309-313). In addition, 5,6-carboxyfluoroscein has been used in vivo to measure the pH of tumor tissue (Mordon et al., Photochem. Photobiol., 1994, 60:274-279.) The phosphorescent oxygen probes Green 2W and Oxyphor R2 have been used to measure the oxygenation of cancerous tissue (Lo et al., Adv. Exp. Med. Biol., 1997, 411:577-583; Wilson et al., Adv. Exp. Med. Biol., 1998, 454:603-609), while the hydrogen peroxide probe 2′-7′-dichlorofluoroscein has been used in vivo to measure the level of oxidative stress (Watanabe, S., Keio J. Med., 1998, 47:92-98).

In another embodiment, probes can be activated by changes in H⁺ ion concentration or pH changes. Probes can be designed to contain spacers that are cleaved when physiological pH values are lowered. Examples of such spacers include alkylhydrazones, acylhydrazones, arylhydrazones, sulfonylhydrazones, imines, oximes, acetals, ketals, and orthoesters.

The methods of analyte activation described herein can be used to detect and/or evaluate many diseases or disease-associated conditions. The redistribution of analytes such as potassium, sodium, and calcium is often indicative of certain physiological processes and diseases including hypoxia and ischemia (e.g., cerebro-vascular ischemia due to stroke, embolism or thrombosis; ischemia of the colon, vascular ischemia due to coronary artery disease of heart disease, ischemia due to physical trauma, poisons, ischemia associated with encephalopathy; and renal ischemia). In addition, tumors are characterized by low pH values by comparison to normal tissue as well as inflammation, particularly inflammation caused by foreign pathogens.

In another embodiment, a quencher molecule is used to quench the initial signal. Prior to activation, the quencher molecule is situated such that it quenches the optical properties of the reporter molecule (i.e., chromophore). Upon activation, the reporter molecule is de-quenched. By adopting these activated and unactivated states in a living animal or human, the reporter molecule and quencher molecule located on the probe will exhibit different signal intensities when the probe is active or inactive. It is therefore possible to determine whether the probe is active or inactive in a living organism by identifying a change in the signal intensity of the reporter molecule, the quencher molecule, or a combination thereof. In addition, because the probe can be designed such that the quencher molecule quenches the reporter molecule when the probe is not activated, the probe can be designed such that the reporter molecule exhibits limited signal until the probe is either hybridized or digested. For example, the quencher DABCYL was utilized to record apoptosis associated caspase-3 activity using a near infrared chromophore (NIRM image at 700 NM). There was a significantly lower signal when caspace-3 inhibitor was present.

There are a number of quenchers available and known to those skilled in the art including, but not limited to, DABCYL, QSY-7 (Molecular probe), QSY-33 (Molecular probe), Fluorescence dyes such as Cy5 and Cy5.5 pare (Schobel, Bioconjugate 1999, 10, 11107), Fluorescein Isothiocyanates (FITC) and Rhodamine pair (Molecular Probes, Inc., OR).

An additional method of detection includes two distinct fluorochromes (fluorochrome1 and fluorochrome2) that are spatially near one another such that fluorescent resonance energy transfer (FRET) takes place. Thus, initially, excitation at the fluorochrome1 excitation wavelength results in emission at the fluorochrome2 emission wavelength secondary to FRET. Activation of the probe can be determined in this embodiment as loss of signal at the fluorochrome2 emission wavelength with excitation at fluorochrome1 excitation wavelength. Signal increase at the fluorochrome1 emission wavelength after excitation at the fluorochrome1 excitation wavelength may aide the determination of activation in this case. Emission at the fluorochrome2 emission wavelength after excitation at the fluorochrome2 wavelength can also be used to determine local probe concentration.

Alternatively, the FRET method can be used to determine activation of probes when two components are brought into proximity after enzymatic activity (e.g., ubiquination), such that fluorochrome1 and fluorochrome2, which are initially spatially separated, are subsequently spatially near enough to each other so that FRET can take place. Thus, activation is detected by exciting at the fluorochrome1 excitation wavelength and recording at the fluorochrome2 emission wavelength.

In Vitro Probe Testing

After an imaging probe is designed and synthesized, it can be tested routinely in vitro to verify a requisite level of signal before activation. Preferably, this is done by obtaining a signal value for the quenching, de-quenching, wavelength shift, fluorescence energy transfer, fluorescence life time change, polarity change of the fluorochrome-containing probe, etc. in a dilute, physiological buffer. This value is then compared to the signal value obtained from an equimolar concentration of free chromophore in the same buffer, under the same chromophore-measuring conditions. Preferably, this comparison will be done using a series of dilutions, to verify that the measurements are taking place on a linear portion of the signal value vs. chromophore concentration curve.

The molar amount of a chromophore on a probe can be determined by one of ordinary skill in the art using any suitable technique. For example, the molar amount can be determined readily by near infrared absorption measurements. Alternatively, the molar amount can be determined readily by measuring the loss of reactive linking groups on the backbone or spacer, e.g., decrease in ninhydrin reactivity due to loss of amino groups.

In another procedure, the chromophore signal emittance is measured before and after treatment with an activating agent, e.g., an enzyme. If the probe has activation sites in the backbone (as opposed to in spacers), de-quenching should be tested at various levels of chromophore loading, where “loading” refers to the percentage of possible chromophore linkage sites on the backbone actually occupied by chromophores.

In addition, cells grown in culture can be used routinely to test the imaging probes of the present invention. Probe molecules free in cell culture medium should be non-detectable by fluorescence microscopy. Cellular uptake should result in probe activation and a fluorescence signal from probe-containing cells. Microscopy of cultured cells thus can be used to verify that activation takes place upon cellular uptake of a probe being tested. Microscopy of cells in culture is also a convenient means for determining whether activation occurs in one or more subcellular compartments.

It will be appreciated that the compositions and methods of the present invention may be used in combination with other imaging compositions and methods. For example, the methods of the present invention may be used in combination with traditional imaging modalities such as CT, PET/SPECT or MRI, and probes used in these methods can contain components, such as iodine, gadolinium atoms or radioactive isotopes, which change imaging characteristics of tissues when imaged using CT, PET, SPECT, or MR. For example, the probes of the present invention may be constructed using a plurality of chromophores chemically linked to chromophore attachment moieties with various magnetic properties, such as crosslinked iron oxide nanoparticle (CLIO). These dual optical/MR imaging probes can be used for imaging not only the molecular activity of a variety of different enzymes by measuring fluorescence activation, but also their precise localization from their effects on T2 weighted MR images.

Further, it will be appreciated that the imaging methods of the present invention can be combined with therapeutic methods. For example, if the probes of the present invention detect a tumor, an immediate anti-tumor therapy can be employed. Moreover, the probes themselves can contain a component that is therapeutic or becomes therapeutic after target interaction.

In Vivo Near Infrared Imaging

Although the invention involves novel imaging probes, general principles of fluorescence, optical image acquisition, and image processing can be applied in the practice of the invention. For a review of optical imaging techniques, see, e.g., Alfano et al., 1997, “Advances in Optical Imaging of Biomedical Media,” Ann. NY Acad. Sci., 820:248-270.

An imaging system useful in the practice of this invention typically includes three basic components: (1) a near infrared light source, (2) a means for separating or distinguishing emissions from light used for chromophore excitation, and (3) a detection system.

The light source provides monochromatic (or substantially monochromatic) near infrared light. The light source can be a suitably filtered white light, i.e., bandpass light from a broadband source. For example, light from a 150-watt halogen lamp can be passed through a suitable bandpass filter commercially available from Omega Optical (Brattleboro, Vt.). In some embodiments, the light source is a laser. See, e.g., Boas et al., 1994, Proc. Nat. Acad. Sci. USA 91:4887-4891; Ntziachristos et al., 2000, Proc. Natl. Acad. Sci. USA 97:2767-2772; Alexander, 1991, J. Clin. Laser Med. Surg. 9:416-418. Information on near infrared lasers for imaging can be found at http://www.imds.com and various other well-known sources.

A high pass or bandpass filter (700 nm) can be used to separate optical emissions from excitation light. A suitable high pass or bandpass filter is commercially available from Omega Optical. In the case of quantum dots, a single excitation wavelength can be used to excite multiple different fluorochromes on a single probe or multiple probes (with different activation sites), and spectral separation with a series of bandpass filters, diffraction grating, or other means may be used to independently read the different activations.

In general, the light detection system can be viewed as including a light gathering/image forming component and a light detection/image recording component. Although the light detection system may be a single integrated device that incorporates both components, the light gathering/image forming component and light detection/image recording component will be discussed separately. However, a recording device may simply record a single (time varying) scalar intensity instead of an image. For example, a catheter-based recording device can record information from multiple sites simultaneously (i.e., an image), or may report a scalar signal intensity that is correlated with location by other means (such as a radio-opaque marker at the catheter tip, viewed by fluoroscopy).

A particularly useful light gathering/image forming component is an endoscope. Endoscopic devices and techniques that have been used for in vivo optical imaging of numerous tissues and organs, including peritoneum (Gahlen et al., 1999, J. Photochem. Photobiol. B 52: 131-135), ovarian cancer (Major et al., 1997, Gynecol. Oncol. 66:122-132), colon (Mycek et al., 1998, Gastrointest. Endosc. 48:390-394; Stepp et al., 1998, Endoscopy 30:379-386) bile ducts (Izuishi et al., 1999, Hepatogastroenterology 46:804-807), stomach (Abe et al., 2000, Endoscopy 32:281-286), bladder Kriegmair et al., 1999, Urol. Int. 63:27-31; Riedl et al., 1999, J. Endourol. 13:755-759), and brain (Ward, 1998, J. Laser Appl. 10:224-228) can be employed in the practice of the present invention. FIG. 4 shows a schematic representation of an endoscope for use with in new methods and probes.

Other types of light gathering components useful in the invention are catheter-based devices, including fiber optics devices. Such devices are particularly suitable for intravascular imaging. See, e.g., Tearney et al., 1997, Science 276:2037-2039; Proc. Natl. Acad. Sci. USA 94:4256-4261.

Still other imaging technologies, including phased array technology (Boas et al., 1994, Proc. Natl. Acad. Sci. USA 91:4887-4891; Chance, 1998, Ann. NY Acad. Sci. 838:29-45), diffuse optical tomography (Cheng et al., 1998, Optics Express 3:118-123; Siegel et al., 1999, Optics Express 4:287-298), intravital microscopy (Dellian et al., 2000, Br. J. Cancer 82:1513-1518; Monsky et al, 1999, Cancer Res. 59:4129-4135; Fukumura et al., 1998, Cell 94:715-725), and confocal imaging (Korlach et al., 1999, Proc. Natl. Acad. Sci. USA 96:8461-8466; Rajadhyaksha et al., 1995, J. Invest. Dermatol. 104:946-952; Gonzalez et al., 1999, J. Med. 30:337-356) can be employed in the practice of the present invention. Any diffuse optical tomographic technique, including but not limited to continuous wave, pulsed light, time of flight, early arriving photon methods may be used with the present invention.

Any suitable light detection/image recording component, e.g., charge coupled device (CCD) systems, photomultiplier tubes, or photographic film, can be used in the invention. The choice of light detection/image recording will depend on factors including type of light gathering/image forming component being used. Selecting suitable components, assembling them into a near infrared imaging system, and operating the system is within ordinary skill in the art.

In some embodiments of the invention, two (or more) probes containing: (1) chromophores that emit optical signals at different rear infrared wavelengths, and (2) activation sites recognized by different enzymes, e.g., cathepsin D and MMP2, are used simultaneously. This allows simultaneous evaluation of two (or more) biological phenomena.

In some embodiments of the invention, an additional chromophore that emits light at a different near infrared wavelength is attached to the probe that is not in an optical-quenching interaction-permissive position. Alternatively, two chemically similar probes, one activatable and one non-activatable, each labeled with a different chromophore, can be used. By using the ratio of activatable and non-activatable probe fluorescence, the activity of enzymes can be determined in a manner which is corrected for the ability of tissues to accumulate variable amounts of these probes. Both of these approaches can be used to monitor delivery of the probe, to track the probe, to calculate doses, and to serve as an internal standard for calibration purposes.

Pharmaceutically acceptable carriers, adjuvants, and vehicles may be used in the composition or pharmaceutical formulation of this invention. Included carriers, adjuvants, or and vehicles include, but are not limited to, ion exchangers, alumina, aluminum stearate, lecithin, serum proteins such as albumin, buffer substances such as phosphate, glycine, sorbic acid, potassium sorbate, TRIS (tris(hydroxymethyl)amino methane), partial glyceride mixtures of fatty acids, water, salts or electrolytes, disodium hydrogen phosphate, potassium hydrogen phosphate, sodium chloride, zinc salts, colloidal silica, magnesium trisilicate, polyvinyl pyrrolidone, cellulose-based substances, polyethylene glycol, sodium carboxymethylcellulose, polyacrylates, waxes, polyethylene-polypropylene block polymers, sugars such as glucose, and suitable cryoprotectants.

The pharmaceutical compositions of the invention may be in the form of a sterile injectable preparation. This preparation can be prepared by those skilled in the art of such preparations according to techniques known in the art. The possible vehicles or solvents that can be used to make injectable preparations include water, Ringer's solution, and isotonic sodium chloride solution, and D5W. In addition, oils such as mono- or di-glycerides and fatty acids such as oleic acid and its derivatives can be used.

The probes and pharmaceutical compositions of the present invention can be administered orally, parentally, by inhalation, topically, rectally, nasally, buccally, vaginally, or via an implanted reservoir. The term “parental administration” includes intravenous, intramuscular, intra-articular, intra synovial, intrasternal, intrathecal, intraperitoneal, intracisternal, intrahepatic, intralesional, and intracranial injection or infusion techniques. The probes may also be administered via catheters or through a needle to any tissue.

For ophthalmic use, the pharmaceutical composition of the invention may be formulated as micronized suspensions in isotonic, pH adjusted sterile saline. Alternatively, the compositions can be formulated in ointments such as petrolatum.

For topical application, the new pharmaceutical compositions can also be formulated in a suitable ointment, such as petrolatum. Transdermal patches can also be used. Topical application for the lower intestinal tract or vagina can be achieved by a suppository formulation or enema formulation.

The formulation of the probe can also include an antioxidant or some other chemical compound that prevents or reduces the degradation of the baseline fluorescence, or preserves the fluorescence properties, included but not limited to, quantum yield, fluorescence lifetime, excitation and emission wavelengths. These antioxidants or chemical compounds may include, but are not limited to, melatonin, dithiotreitol (dTT), defroxamine (DFX), methionine and N-acetyl cysteine.

Dosing of the invention will depend on a number of factors including instrumentation sensitivity as well as a number of subject-related variables including animal species, age, body weight, mode of administration, sex, diet, time of administration, and rate of excretion.

Prior to use of the invention or any pharmaceutical composition of the invention, the subject may be treated with an agent or regimen to enhance the imaging process. For example, a subject may be put on a special diet prior to imaging to reduce any autofluorescence or interference from ingested food, such as a low pheophorbide diet to reduce interference from fluorescent pheophorbides that are derived from some foods, such as green vegetables. Alternatively, a cleansing regimen may be used prior to imaging, such as those cleansing regimens that are used prior to colonoscopies and include use of agents such as Visiciol.

The subject (patient or animal), may be treated with pharmacological modifiers to improve image quality. For example—with low dose enzymatic inhibitors to decrease background signal relative to target signal (secondary to proportionally lowering enzymatic activity of already low-enzymatic activity normal tissues to a greater extent than enzymatically-active pathological tissues) may improve the target to background ratio during disease screening. As another non-limiting example pretreatment with methotrexate to relatively increase uptake in abnormal tissue (i.e., metabolically active cancers), with folate based targeted delivery may be employed.

### EXAMPLES

In order that the invention may be more fully understood, the following examples are provided. It should be understood that these examples are for illustrative purposes only and are not to be construed as limiting the invention in any way.

I. Synthesis of Near Infrared Fluorescence Probes

Several different intramolecularly-quenched near infrared imaging probes were synthesized by conjugating a commercially-available fluorochrome known as Cy5.5 (absorption=675 nm, emission=694 nm; Amersham, Arlington Heights, Ill.) to PL-MPEG (average molecular weight approx. 450 kD). The three probes differed in attachment of the fluorochrome to the polylysine backbone. In a probe designated “Cy-PL-MPEG,” the Cy5.5 was linked directly to the ε-amino group of the polylysine side chains at various densities, which ranged from 0.1% to 70% derivatization of the E-amino groups. In a probe designated, “Cy-RRG-PL-MPEG,” the Cy5.5 fluorochrome was linked to the polylysine by a spacer consisting of Arg-Arg-Gly. In a probe designated “Cy-GPICFFRLG-PL-MPEG,” the Cy 5.5 fluorochrome was linked to the polylysine by a spacer consisting of Gly-Pro-Ile-Cys-Phe-Phe-Arg-Leu-Gly (SEQ ID NO:1). Trypsin and trypsin-like proteases are capable of cleaving the polylysine backbone of Cy-PL-MPEG, when it is only partially derivatized.

Probes Cy-RRG-PL-MPEG and Cy-GPICFFRLG-PL-MPEG were designed to allow fluorochrome cleavage of the spacer, but not necessarily the backbone. For example the peptide spacer RRG, sensitive to trypsin cleavage, was used to derivatize the PL-MPEG, and then Cy5.5 was linked to the N-terminus of the RRG spacers. The cathepsin D sensitive peptide spacer, GPICFFRLG (SEQ ID NO:1), was similarly used to derivative the PL-MPEG.

Cy5.5, commercially available as a monofunctional NHS-ester (Amersham, Arlington Heights, Ill.), was used according to the vendor's instructions, to label free ε-amino groups of the polylysine backbone in PL-MPEG. Cy5.5 was added to a pre-mixed MPEG-PL solution (0.2 mg PL-MPEG in 1 ml 80 mM sodium bicarbonate solution) to a final concentration of 17 μM. After three hours, the reaction mixture was applied to a Sephadex® G-25 (Pharmacia) column (12 cm) for separation of the reaction product (Cy-PL-MPEG) from the unreacted fluorochrome and other low-molecular weight components of the reaction mixture. Average fluorochrome loading was about 20%, i.e., 11 out of 55 free amino groups on the PL-MPEG labeled with Cy5.5 (based on TNBS assay and absorption measurement).

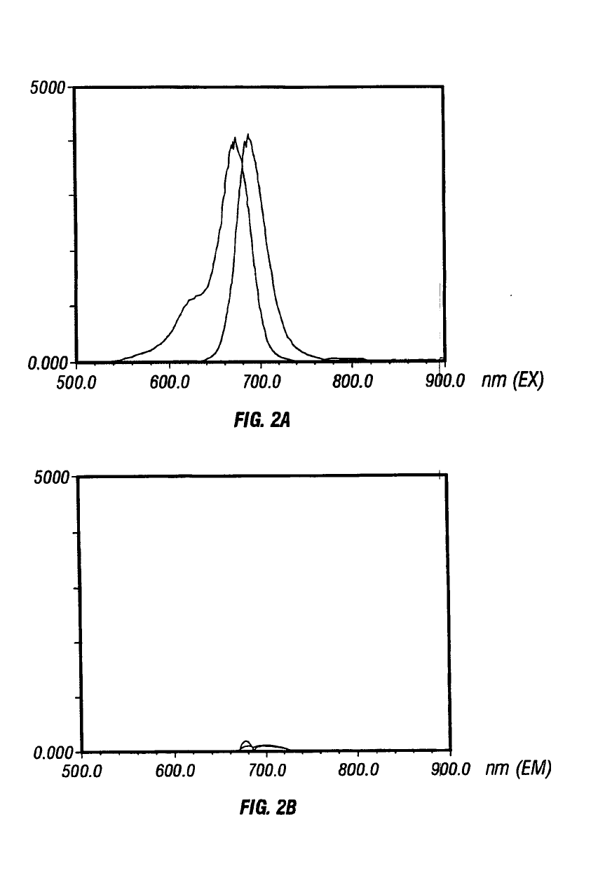

FIG. 2A shows the excitation and emission spectra of Cy5.5 free in solution. FIG. 2B shows the excitation and emission spectra of Cy5.5 fluorochrome of Cy-PL-MPEG. The excitation and emission wavelengths of Cy5.5 are 675 nm and 694 nm, respectively. There was a marked difference in the level of fluorescence of the free Cy5.5 and the Cy-PL-MPEG. The fluorescence level of the Cy-MPEG-PL was approximately 30-fold lower than that of the unbound Cy5.5.

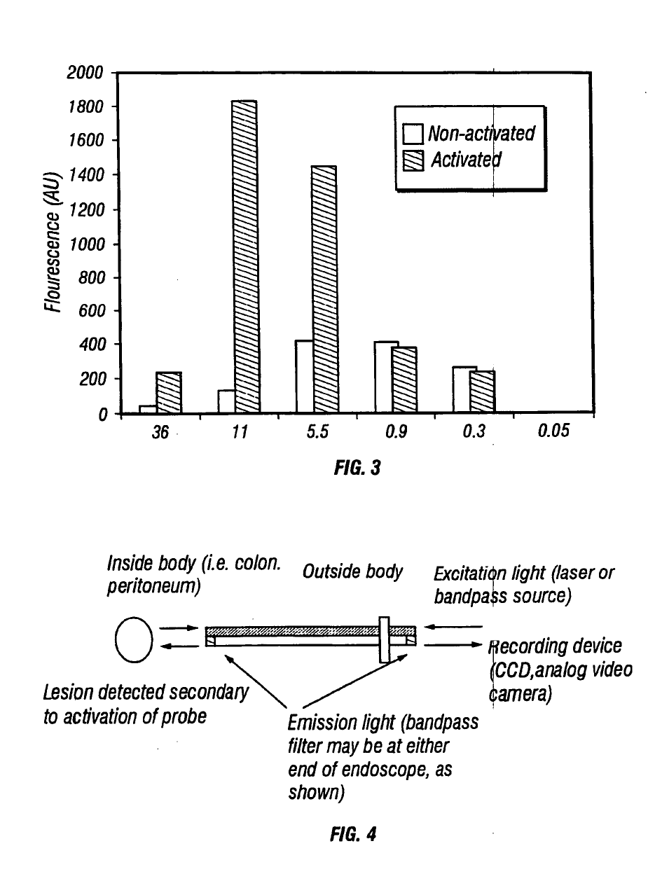

In subsequent studies, we determined the effect of chromophore loading (i.e., percentage of ε-amino groups on the polylysine backbone occupied by chromophore) on the optical properties of the probe. FIG. 3 shows the relative fluorescent signal of Cy(n)-MPEG-PL (white bars) as a function of percentage of ε-amino groups on the polylysine backbone occupied by fluorochrome. At 20% loading (11 of 55 groups) and higher, intramolecular quenching was observed, and the fluorescence signal was lowered in comparison to probes with lower fluorochrome loading. After trypsin cleavage of the backbone, fluorescence signal was recovered, as shown by the black bars in FIG. 3. Maximum fluorescence recovery was obtained at 20% loading (15-fold fluorescence signal increase upon activation). Recovery was reduced when loading was greater than 20%. This may have been due to steric hindrance and the need for free lysine groups for efficient cleavage of the backbone.

II. Probe Activation in Cell Culture

The next step in testing the imaging probe was to perform cell culture experiments. We expected that non-internalized Cy-PL-MPEG would be non-detectable by fluorescence microscopy, and that cellular uptake would lead to activation of the probe, with a resulting fluorescence signal. Data obtained using amelanotic B16 melanoma cells confirmed our prediction and showed that: (1) the non-activated probe is non-fluorescent, (2) the probe is taken up by this cell line, and (3) cellular uptake results in activation of the probe and fluorescence signal detection.

In this experiment we compared a bright field image outlining B16 cells to: (1) the same field under near infrared fluorescence conditions when Cy-MPEG-PL was added to the cells, near time-zero; and (2) after allowing time for intracellular uptake of the probe (data not shown). The cells were not detectable by near infrared fluorescence near time-zero, but the cells were clearly visible (due to intracellular fluorescence) after cellular uptake of the probe, i.e., at about two hours. This experiment demonstrated that our imaging probe detectably changed its optical properties in a target cell-dependent manner.

III. In Vivo Imaging

We used an imaging system composed of three main parts: light source, platform/holder, and image recording device to perform our in vivo imaging studies. A fiber optic light bundle with a 150 W halogen bulb (Fiberlite high intensity illuminator series 180, Dolan-Jennen Industries) provided broad spectrum white light. A sharp cut off band pass optical filter (Omega Filter Corp., Brattleboro, Vt.) was mounted at the end of the fiber optic bundle to create a uniform excitation source in the 610-650 nm range. The light was placed approximately 15 cm above the imaging platform to provide homogenous illumination of the entire mouse. The platform itself was a matte black surface that decreased the number of excitation photons reflected (and possibly detected) by the recording device.

Fluorescent (emission) photons were selected using a low pass filter with a sharp cut off at 700 nm (Omega Filter Corp.), although as stated above, laser sources and/or bandpass emission filters may alternatively be employed. Cy5.5 dye has an excitation peak at approximately 670 nm, with a broad shoulder extending below 610 nm. Peak emission is at 694 nm. Sharp cut-off filters with more than 5 OD attenuation combined with widely spaced frequencies for the filter set markedly decreased “cross talk” of incident excitation photons recorded as fluorescent emission signal. The narrow angle between light source and recording device ensured that only fluorescent emission photons or scattered photons that interacted with the mouse tissue reached the low pass filter.

For image recording, the low-pass filter was mounted on a low power microscope (Leica StereoZoom 6 photo, Leica microscope systems, Heerbrugg, Switzerland). A low light CCD (SenSys 1400, 12 bit cooled CCD, Photometrics, Tucson, Ariz.) recorded the fluorescent emission images. Images were transferred to a PowerMac 7600/120 PC (Apple Computer, Cupertino, Calif.) and processed using IPLab Spectrum 3.1 software (Signal Analytics Corp., Vienna, Va.). Post processing included standard routines to exclude bad CCD pixels, and superimposition routines to overlay emission images with localization images of the entire mouse obtained using a second white light source. Typical acquisition time was 30 seconds for the near infrared emission images, and 1 second for the white light (non-selective images).

### Example 1

To demonstrate the ability of the probes to image tumors, we tested the near intramolecularly-quenched infrared imaging probe (Cy₁₁-PL-MPEG; 20% fluorochrome loading) in tumor-bearing mice. Nude mice bearing tumor line 9L or LX1 received 2 nmol of Cy₁₁-PL-MPEG intravenously. The mice were imaged by near infrared light immediately and up to 36 hours after intravenous administration of the probe. The tumor was visible as an area of intense fluorescence, in contrast to the surrounding tissue. An increase in fluorescence signal within tumor was observed as a function of time, as the probe was internalized into tumor cells and became activated by endosomal hydrolases.

Using cathepsin D (2000, Cancer Res. 60: 4953-4958) as a model target protease, we synthesized a long circulating, synthetic graft copolymer bearing near infrared (NIR) fluorochrome positioned on cleavable substrate sequences. In its native state, the reporter probe was essentially non-fluorescent at 700 nm due to energy resonance transfer among the bound fluorochrome. NIR fluorescence signal activation was linear over at least four orders of magnitude and specific when compared to scrambled nonsense substrates. Using matched rodent tumor model cells implanted into nude mice expressing or lacking the targeted protease, it could be shown that the former generated sufficient NIR signal to be directly detectable and that signal was significantly different compared to negative control tumors. Representative optical images of the lower abdomen of a nude mouse implanted with a CaD+ and CaD− tumor were taken. The CaD+tumor emits fluorescence while the CaD− tumor has a significantly lower signal. A thresholded false color map can be generated by superimposing a white light image with a fluorescence image.

The present invention may therefore be useful in detecting and evaluating cancers, and delineating tumor margins, wherein the probe is directed to tumor tissue. Detection methods include, but are not limited to, reflective devices such as endoscopes, cameras, infrared goggles, and operating microscopes; and diffuse optical tomographic devices such as employed in Ntziachristos et al., 2000, Proc. Natl. Acad. Sci. USA 97:2767-2772. A partial list of tumors include, but are not limited to tumors of the breast, prostate, colon, bronchi, lung, brain, ovary, muscle, fat, esophagus, head and neck, skin, small bowel, stomach, liver, adrenal gland, kidneys, bladder, pancreas, bone, ureters, blood vessels, and resultant metastases to lymph nodes and elsewhere.

### Example 2

To demonstrate the ability of fluorescent probes to image colonic polyps, malignant and benign Apc-Min (C57BL/6J-Apc^{Min}) mice, a strain highly susceptible to spontaneous intestinal adenoma formation, were evaluated after the intravenous injection of 2 nmol per mouse of cathepsin B sensitive probe. Twenty-four hours after probe injection, animals were sacrificed and colons resected. White light and fluorescent images demonstrated the marked difference in fluorescent signal intensity in the polyps as compared to adjacent normal epithelium.

The resulting marked increase in contrast between normal and abnormal tissue may be exploited during colonoscopy (or endoscopy) to aid in lesion detection.

### Example 3

To demonstrate the ability of the probes of the current invention to image ovarian cancer, very small peritoneal tumor deposits using CaD− and CaD+ cell lines (transfected 3Y1 rat embryonic tumor cell line) were implanted into mice intraperitoneally. The Cathepsin D probe described in more detail previously was then administered IV and the peritoneal surfaces were imaged 24 hours later using white light (i.e. as in conventional endoscopy) or at 700 nm (NIRF imaging). Microscopic deposits of 300 μm could be readily detected by NIRF imaging that were not visible by white light imaging.

The resulting marked increase in detection of minimal residual disease in ovarian cancer may be exploited during laproscopy (or endoscopy) to aid in lesion detection and to monitor therapy.

### Example 4

To demonstrate the ability of the probes to image atherosclerosis, especially active or vulnerable plaques, control mice (C57BL/6) and Apoe-deficient (C57BL/6J-Apoe^{tm/Une}) mice, which spontaneously develop arterial fatty streaks and atheromatous plaques, were evaluated after the intravenous injection of 2 nmol per mouse of a cathepsin B-sensitive probe. Twenty-four hours after probe injection, animals were sacrificed, and aortas were resected in toto from aortic root to beyond the iliac bifurcation. Using the previously described imaging system, white light and NIR fluorescent images of control and ApoE Mouse aortas were acquired. Plaque burden, as well as degree of plaque activity, was revealed in the fluorescent images, and was markedly different in control (minimal fluorescence) and ApoE mice (highly fluorescent). Fluorescent images were acquired under identical conditions, and were displayed using identical brightness parameters.

The present invention may therefore be useful in detecting and evaluating cardiovascular disease and helping guide surgical interventions, wherein the probe is directed to vascular tissue.

One method of administering the probes of the present invention to vascular tissue is via catheters or by disruption of probe-containing microbubbles by local deposition of resonant energy at ultrasound frequencies, both well known procedures.

### Example 5

To demonstrate the ability of the probes to image inflammatory (rheumatoid) arthritis, arthritic and non-arthritic littermates were evaluated after the intravenous injection of 2 nmol per mouse of cathepsin B sensitive NIRF probe. The K/BxN T cell receptor (TCR) transgenic mouse line, derived from a cross of KRN/C57B 1/6 TCR with the NOD strain (Matsumoto, et. al., Science, 286:1732-1735 (1999)), which develops a disease very similar to human rheumatoid arthritis in 50% of animals, while 50% of animals remain unaffected, was used. White light and fluorescent images were acquired 24 hours after probe injection. The foot of a non-arthritic mouse and of an arthritic mouse demonstrate: 1) the marked overall increased fluorescent signal intensity in affected joints in arthritic animals, and 2) the non-invasive visualization of the heterogeneous distribution of phenotypic (clinical) disease in inflammatory arthritis.

Probes of different polymer lengths were also used. An approximately 120 kD cathepsin B sensitive probe was injected into arthritic mice. Fluorescent imaging at 24 hours again revealed the marked heterogeneity in distribution of disease, in this case between right and left feet in the same animal.

The present invention may therefore be useful in detecting and evaluating inflammatory diseases such as rheumatoid arthritis, wherein the probe is directed to inflammation. It may also be useful for measuring therapeutic efficacy against such diseases.

One method of administering the probes of the present invention to arthritis areas is via intrarticular injections, a well known procedure.

### Example 6

Imaging of specific enzymes in osteoporosis development and its treatment are useful for drug development and/or clinical use. Several proteases have been implicated in osteoporosis development, in particular cathepsin K, which is produced by osteoclasts. Numerous osteoclast inhibitors are in clinical use. Specific peptide substrates for cathepsin K that can be utilized in the probes of the present invention include, but are no limited to, Z-Leu-Arg-AMC, Z-Pro-Arg-AMC, Z-Phe-Arg-AMC, and Z-Phe-Arg-pNA ((1999) Biochemistry, 38:13594-13583; (2000) Biochemistry, 39:529-536).

### Example 7

To illustrate the ability of the probes of the present invention to image thrombosis, a thrombin probe was synthesized. The design of the protease activatable NIRF probe was based on a long circulating graft copolymer as a delivery vehicle, the peptide substrate and a near-infrared fluorochrome. The biological fate of the long circulating polymer (a partially pegylated polylysine copolymer) has been extensively studied in animals and humans. The circulation time of the polymer is over 20 hours in human and is thus ideally suited for vascular imaging application. We started out by attaching the peptide substrate to unpegylated lysine residues of the polymer. The synthesized 11-amino-acid peptide, Gly-D-Phe-Pip-Arg-Ser-Gly-Gly-Gly-Gly-Lys(FITC)-Cys-NH2, was designed to contain a thrombin sensitive substrate, a tetraglycine spacer, a fluorescein tag for quantitation and a cysteine residue for further conjugation. The thrombin substrate sequence, D-Phe-Pip-Arg, had a D-phenylanaline at the P3 position and an unusual amino acid pipecolic acid at the P2 position. The substrate has a reported k_{cat}/K of 3.94×10₇ M₋₁S₋₁.

We first performed an enzymatic assay to show that the fully designed, C-terminal extended peptide still served as a substrate for thrombin. Using HPLC, we found that the peptide was recognized by thrombin and cleaved into two major products. In contrast, there was no cleavage when the serine at P1′ position was replaced by a proline residue. The control peptide, Gly-D-Phe-Pip-Arg-Pro-Gly-Gly-Gly-Gly-Lys(FITC)-Cys-NH₂, remained intact for two hours following incubation with thrombin.

The peptide was coupled to the polymer (PGC) using biofunctional iodoacetic anhydride as the connecting linker. The unpegylated free amino groups on the PGC backbone were capped with iodoacetic anhydride, converting all amino groups into thiol reactive groups, which were subsequently reacted with peptides. In the final step of synthesis, monoreactive indocyanine fluorochrome (Cy5.5) was conjugated to the Nterminus of each peptide. On average, each polymer molecule contained 23 reporter substrate/fluorochromes. With this high number of reporters, fluorescence was efficiently quenched in the inactivated state. Similar conjugation efficiency and optical characteristics were obtained for the control probe.

The prepared probes were first tested with purified thrombin in PBS buffer as the NIRF signal was recorded over time. Initially both probes showed low NIR fluroescence (150 arbitrary units (AU)) (FIG. 3A). Following addition of thrombin, NIRF signal increased from 150 AU to 4100 AU within 20 minutes (27 fold increase). This was significantly greater activation compared to the control probes, with only a 1-fold increase in NIRF signal within the same time frame. There was a clear dose response when the probe was incubated with different amounts of thrombin. To further demonstrate the specificity of thrombin-activation, we examined probe activation in the presence of hirudin, a direct thrombin inhibitor used in the clinical treatment of vascular thrombosis. When thrombin was added to solutions containing the thrombin probe and hirudin, significantly less NIRF signal was detected compared to hirudin-free solutions. Furthermore, to show that hirudin did not destroy or alter the optical probe, we added additional thrombin, which overcame hirudin activation, releasing a strong NIRF signal.

An imaging experiment was subsequently carried out to confirm that thrombin activated the thrombin probe but not other enzyme specific probes. A home-built imaging system which has a bandpass excitation filter at 610-650 nm and an emission filter at 680-720 nm was used to acquire NIRF image of activation with various probes. Thrombin, control, cathepsin B and cathepsin D probes were incubated with thrombin, individually. The NIRF and bright field images were acquired 10 min after incubation. Without thrombin, there was no detectable fluorescent signal in any of the probes. Within 10 min after thrombin addition however, NIR fluorescence signal was selectively generated by the thrombin probe.

To demonstrate thrombin-activation of the probe in human blood, citrated human whole blood was incubated with the thrombin probe and NIR fluorescence was recorded. There was no detectable NIRF signal within 30 min of incubation of the probe in anticoagulated blood. Following exogenous thrombin addition, NIRF signal increased within minutes as visual blood clotting was. Interestingly, as shown in FIG. 4B, the NIRF signal further increased slowly over time. Compared to the probe experiments in buffer, this finding may be due to restricted mixing of the target probe with thrombin in the semi-solid blood clot. Exogenous thrombin was necessary to generate the NIRF signal, suggesting that the anticoagulant effects of sodium citrate inhibited endogenous thrombin generation.

Thrombosis is a central pathophysiologic feature of many cardiovascular diseases such as unstable angina and myocardial infarction, as well as deep venous thrombosis and pulmonary embolism. Rapid diagnosis of these potentially life-threatening conditions is necessary to minimize the associated morbidity and mortality. Current diagnostic imaging methods are flowbased (x-ray angiography, computed tomography angiography, magnetic resonance angiography, doppler ultrasound) or perfusion-based (nuclear medicine perfusion scans) and suffer from two important limitations. First, these methods do not directly image thrombus, and therefore cannot reliably distinguish between a thrombotic or nonthrombotic (e.g. cholesterol, lipid) obstruction to flow. Second, these methods do not allow assessment of biological regulation of thrombus formation.

The results indicate that the developed probes have the potential to serve as imaging reporters for thrombus activation in vivo and biological studies in animal models are currently ongoing. Three-dimensional tomographic imaging systems could be used with a thrombin probe to allow quantitative imaging of probe activation in deep tissue in vivo. This targeted optical imaging technology may ultimately contribute to the understanding, diagnosis, and treatment of vascular thrombosis.

### Example 8

The paradigm of activatable probe imaging can be extended to multiple wavelengths, to probe different tissue and enzyme characteristics in vivo simultaneously. Nude mice were implanted with 9L tumors or 9L tumors stably transfected to overexpress green fluorescent protein (GFP). Twenty four hours after the intravenous injection of 2 nmol per mouse of cathepsin B sensitive probe, mice were imaged using white light, filter combinations sensitive to the cathepsin B probe, and filter combinations sensitive to GFP fluorescence. By reviewing the cathepsin B and GFP images, one can obtain a ratio image of the GFP image divided by the cathepsin B image. The difference in relative gene expression levels between the two tumors (GFP and cathepsin B expression), are revealed in this ratio image, which illustrates the utility of multi-channel imaging.

The major advantages of imaging different biological targets simultaneously and independently include the ability to 1) co-localize targets, 2) probe for differential expression levels of multiple targets, 3) analyze the combination of expression levels of particular importance in cancer, where one target alone is rarely overexpressed, 4) develop mini-arrays for in vivo target assessment, 5) image the temporal and spatial correlation of distinct biological pathways in disease, and 6) image the effects of therapy on different biological targets simultaneously, and 7) evaluate tissue characteristics by exogenous probe administration combined with intrinsic chromophore gene expression, such as intrinsic bioluminescence (i.e., tissues transfected to express luciferase) with exogenous activatable probe administration.

### Example 9

The following example illustrates the ability of the probes of the present invention to image to identify the efficacy of therapeutic drug candidates and measure a dose response and to assess drug levels in a subject.

The synthesized probes contain a preferential MMP-2 peptide substrate. Two different peptide substrates were used in this study, an MMP-2 cleavable peptide (GPLGVRGK(FITC)C-NH₂ (SEQ ID NO:10) (substrate sites are italicized)) and a scrambled control peptide. The ability of MMP-2 to recognize the substrates was initially confirmed by HPLC showing only cleavage of the former but not the latter. Although the latter control probe has a GVR leader sequence, it is too short to be recognized by MMP-2. These results are also in agreement with extensive prior literature on MMP substrate selectivity.

Each assembled reporter molecule contained an average of 12 cleavable proteinase reporter groups conjugated to the N-terminus of the peptide substrate resulting in efficient quenching of the near infrared fluorochrome (<90 AU at 0.3 μM concentrations of Cy 5.5). When the reporter molecules were tested in vitro against purified active MMP-2, fluorescence increased significantly (up to 850%) while there was essentially no change in fluorescence when the control peptide was grafted onto the imaging probe. To confirm that cell-secreted MMP-2 could also activate the probe, we used conditioned medium from fibrosarcoma cells (HT1080) activated with p-aminophenyl mercuric acid (APMA). As in the above studies, NIR fluorescence increased several hundred percent while there was no increase using the control probe with the scrambled peptide. In additional studies we also incubated the probe against a panel of MMP's: MMP-1, MMP-2, MMP-7, MMP-8 and MMP-9. The relative fluorescence increase at equimolar conditions for the different MMP's were (scaled to active MMP-2 set to 100%): MMP-1: 19%, MMP-7: 12%, MMP-8: 28% and MMP-9: 19%.

The increase in near infrared fluorescence following enzyme activation occurred over at least 4 orders of magnitude of enzyme concentration using a constant amount of MMP-2 probe. Furthermore, fluorescence activation could be completely blocked by 1 mM of 1,10 phenanthroline, a broad-spectrum experimental MMP inhibitor that acts as a Zinc chelator. To test the probes against more clinically relevant inhibitors, we chose an MMP inhibitor that potently inhibits critical MMPs, such as MMP-2, MMP-3, MMP-9, MMP-13 and MMP-14, at picomolar concentrations. Using 5 U of MMP-2 and 19 pmol of imaging probe, we performed a dose response study of mediated MMP inhibition up to 0.1 mM of inhibitor. At the highest dose tested, the inhibitory effect was 80%. Our estimated Ki was 0.1 nM, similar to the 0.05±0.02 nM value described in the literature. Using other inhibitors, e.g., 1,10 phenanthroline, complete inhibition was observed.

To test the MMP sensitive probe in vivo, the HT1080 human fibrosarcoma tumor model was chosen because of its reported high MMP-2 production and the MMP-2 sensitivity of the developed probe; HT1080 cells also produce MMP-1, MMP-7, MMP-14, MMP-15, and MMP16 and to a lesser degree MMP-9. The BT20 tumor model was chosen because of its relative lack of MMP-2 (confirmed by RT-PCR). In subsequent experiments, zymography was used to probe for MMP-2 activity. These experiments confirmed enzymatic activity both in conditioned medium as well as in tumor tissue (435 U MMP-2/g tumor tissue) of HT1080 cells. In further validation studies we injected either the MMP sensitive probe or the control probe into HT1080 or BT20 tumor bearing animals (the latter serving as another control of a low MMP-2 producing tumor). The imaging results show considerable differences between HT1080 bearing mice injected with the specific (85.0±5.1 AU) or the control probe (27.5±6.6 AU, p<0.001). Furthermore, the MMP devoid BT20 tumors yielded a significantly lower fluorescence signal compared to the HT1080 tumors when imaged with the MMP-2 sensitive probe (31.0±6.6 vs. 85.0±5.1 AU, p<0.001).

We implanted HT1080 tumors into nude mice and grew them to 2-3 mm in size. Animals were then treated with the chosen MMP inhibitor discussed above, or control vehicle, and were then imaged 2 hours after probe administration. It was readily visible from the raw data that there was significantly less MMP-2 NIRF signal in treated tumors when compared to untreated tumors. The differences in MMP-2 NIRF signal among the two groups were statistically significant (39.3±3.7 AU vs. 98.3±5.9 AU, p<0.0001). Indeed, the 2-day treatment reduced tumoral near infrared fluorescence to nearly baseline values observed in previous control experiments.

Probe Synthesis. The MMP-2 peptide substrate Gly-Pro-Leu-Gly-Val-Arg-Gly-Lys(FITC)-Cys-NH₂ (SEQ ID NO:10) (the italicized amino acids correspond to the MMP-2 substrate) and the scrambled control peptide, Gly-Val-Arg-Leu-Gly-Pro-Gly-Lys(FITC)-Cys-NH₂ (SEQ ID NO:13) were synthesized on an automatic peptide synthesizer (PS3, Rainin, Woburn, Mass.) and purified by reverse phase HPLC. The molecular weight of peptides was confirmed by MALDI-MS and was 1275.59 ({M+H}⁺, 1275.45 (calc.)) for the substrate peptide and 1275.96 ({M+H}⁺, 1275.45 (calc.)) for the control peptide. The NIRF probes were prepared according to a previously optimized method in which cathepsin D was targeted. Briefly, a protected graft copolymer (PGC) consisting of a 35 kD poly-Llysine backbone and multiple 5 kD methoxy-polyethylene glycol side chains (MW 500 kD) was reacted with a large excess of iodoacetyl anhydride to convert all remaining amino groups into iodol groups. Specific peptides were then attached to the iodoacetylated PGC through thiol specific reactions. Following conjugation, the monoreactive Cy5.5 dye (Amersham-Pharmacia, Piscataway, N.J.) was attached to the N-terminus of the enzyme peptide substrate. The percent loading of peptide and NIRF dye to PGC was quantitated by absorption measurement using the extension coefficients 250×10³ M⁻¹ cm⁻¹ for Cy 5.5 at 675 nm and 73×10³ M⁻¹ cm⁻¹ for FITC at 494 nm (the latter being attached to the C-terminal lysine). On average, each PGC molecule contained 12 peptide reporter groups containing the terminal cyanine fluorochrome.

Characterization of probe. A number of experiments were conducted to characterize the peptide substrate and imaging probes. Initially we performed HPLC analysis of peptide and control substrates prior to and after incubation with 1 U of MMP-2. One unit is the activity that hydrolyzes 1 μg of type IV collagen within 1 hour using a commercially available assay (gelatinase 72 kD, Boehringer Mannheim, Indianapolis, Ind.). Reverse phase HPLC (Brownlee, Spheri-5, ODS, 30×4.6 mm), using 0.1% TFA and acetonitrile as elution buffers was performed (Rainin Instruments, Woburn, Mass.). To test for the ability of MMP-2 to activate the entirely assembled imaging probe, a constant amount (26.6 pmol of imaging probe corresponding to 320 pmol Cy 5.5) was incubated with 6 U of activated MMP-2 (Boehringer-Mannheim, Ind.; activation was achieved with 2.5 mM of p-aminophenyl mercuric acid; APMA) and fluorescence was determined over time at λₑₓ 675 nm/λₑₘ 694 nm at multiple time points (Hitachi, U4500, Tokyo, Japan). The control probe contained the scrambled peptide. To determine the range of enzyme activation, a constant amount of imaging probe (26.6 pmol) was incubated with variable amounts of activated MMP-2 and fluorescence was determined after 24 hours. Inhibition experiments were performed by incubating 1 U of purified, activated MMP-2 with 19 pmol NIRF probe (220 pmol Cy 5.5) in the presence of different inhibitors, 1,10 phenanthroline (1 mM, Aldrich, Milwaukee. WI), a Zinc chelator, or a direct MMP-2 inhibitor. The latter was also used to inhibit MMP-2 for in vivo studies given its low Kᵢ of 0.05±0.02 nM, bioavailability and the fact that it is being tested in clinical trials.

In additional experiments we tested the MMP sensitive probe against a panel of MMP's. For these experiments we used MMP-1 (human rheumatoid synovial fibroblast, Calbiochem), MMP-2 (human recombinant protein purified from mammalian cells, Calbiochem), MMP-7 (human recombinant, E. Coli, Calbiochem), MMP-8 (human neutrophil granulocyte, Calbiochem) and MMP-9 (human recombinant protein purified from mammalian cells, Oncogene Research Products). Seven pmole of each APMA activated enzyme were incubated for 10 minutes with 10 pmole of the probe at 37° and fluorescence was then determined. Fluorescence activation was scaled to that of APMA activated MMP-2 which was set as 100% (4.7 AU).

Cell culture. HT1080 fibrosarcoma and BT20 mammary adenocarcinoma cells obtained from the American Type Culture Collection (ATCC, Manassas, Va.) were cultured in MEM medium with 2 mM L-glutamine and Earle's BSS adjusted to contain 1.5 g/L sodium bicarbonate, 0.1 mM non-essential amino acids, 1.0 mM sodium pyruvate and 10% heat inactivated fetal bovine serum. Cells were used for zymographic MMP-2 determinations when they were about 60% confluent.

Zymography and RT-PCR. MMP-2 enzyme activity of conditioned medium and tumor tissue was measured by zymography. Briefly, aliquots of the concentrated conditioned medium or tumor homogenate respectively were applied to a 7.5% SDS-PAGE containing 1 mg/ml gelatin. After protein separation, SDS was removed by washing of the gel in 2.5% Triton x-100® (Sigma, St. Louis, Mo.). The gel was then incubated at 37° C. in 50 mM Tris-HCL (pH 7.6) containing 0.2 M NaCl, 5 mM CaCl₂ and 0.02% Brij-35 for 8-16 hours and stained with 1% Coomassie brilliant blue in 30% methanol/10% glacial acetic acid. After de-staining, gelatinolytic activity was visible as a clear band against the blue background. Gels were digitized and enzyme activities were measured against standards of known activity. RT-PCR of HT1080 and BT20 cells was performed using previously published primers for MMP-2.

In vivo studies. Two million cells (either HT1080 or BT20) were injected subcutaneously in the mammary fat pad of athymic nude mice (nu/nu, 5-6 weeks old). Tumors were allowed to grow to 2-3 mm in size. Animals were then anesthetized by an IP injection of ketamine (90 mg/kg) and xylazine (10 mg/kg) and the imaging probe (167 pmol of probe per animal) was injected intravenously. Imaging was typically performed 1-2 hours after IV administration, based on a prior study in which the timing parameters had been optimized. Two different in vivo experiments were performed. In the first experiment we determined the in vivo fluorescence activation in native HT1080 tumors probed with the MMP-2 sensitive probe (n=4), HT1080 tumors probed with the control probe (n=4) or the MMP-2 negative BT-20 tumors imaged with the MMP-2 sensitive probe (n=4). In the second experiment we treated HT1080 tumor bearing animals with either a potent MMP inhibitor (150 mg/kg bid IP for 2 days, n=8 tumors) or with control vehicle (bid IP for 2 days, n=12 tumors). The MMP-2 probe was administered IV 30 minutes after the last of the 4 IP doses of the MMP inhibitor. NIRF imaging was then performed 2 hours after intravenous probe administration. In other experiments animals (n=4) were imaged longitudinally before and after MMP-2 inhibitor treatment initiation.

Imaging. NIRF reflectance imaging was performed using a previously described imaging system. The system consisted of the light-tight chamber equipped with a 150 W halogen white light source and an excitation bandpass filter (610-650 nm, Omega Optical. Brattlebore, Vt.). Light was homogeneously distributed over the field of view (FOV) by light diffusers. Fluorescence was detected by a 12 bit monochrome CCD camera (Kodak, Rochester, N.Y.) equipped with a f/1.2 12.5-75 mm zoom lens and an emission long-pass filter at 700 nm (Omega Optical, Brattlebore, Vt.). Images were acquired over 30 seconds at 610-640 nm excitation and 700 nm emission wavelength. Image analysis was performed using commercially available software (Kodak Digital Science ID software, Rochester, N.Y.). Regions of interest (>200 pixels) were placed over the tumor, the adjacent skin and a reference standard containing 10 nM free Cy 5.5 fluorescent dye imaged in identical position adjacent to each animal. Fluorescence signal was adjusted to this standard and expressed as described previously.

Statistical analysis of different in vivo groups was conducted using an ANOVA-test with Bonferroni correction for multiple comparisons. The treatment effect was tested with a 2-tailed student t-test for paired samples. A p-value smaller 0.05 was considered to be significant. Results are presented as mean±SEM.

Histology. Tumors were excised, fixed for 24 hours in 10% phosphate buffered formalin, paraffin embedded and sectioned into 7 μm slices. Immunohistochemistry was performed using a primary polyclonal goat-antibody against human MMP-2 (Santa Cruz Biotechnology, Santa Cruz, Calif.). An alkaline phosphatase labeled rabbit anti-goat antibody was used to reveal binding of the primary antibody. Endogenous alkaline phosphatase (AP) activity was eliminated by heating (65° C. for 30 minutes) and specific AP activity was visualized using. NBT/BCIP substrate (Boehringer-Mannheim, Ind.). Sections were counterstained with nuclear fast red. Control sections were processed identically however without the primary antibody.

For NIRF fluorescence microscopy tumors were snap frozen and cryosectioned into 8-10 μm slices. Air dried sections were then viewed in phase contrast or fluorescence mode using an inverted epifluorescence microscope (Zeiss Axiovert, Thornwood, N.Y.). Excitation wavelength was 650 nm. A cooled CCD camera (Sensys, Photometrics, Tucson, Ariz.) adapted with a broad band filter (>700 nm) was used for image capture.

The present invention therefore provides compositions and methods for recording native enzyme activities in tumors. This represents an invaluable in vivo tool for elucidation of the functional contribution of specific agents in tumorigenesis, metastagenesis and angiogenesis. Indeed, such measurements can be performed at different resolutions ranging from the microscopic cellular level (e.g., using intravital, confocal, or two photon microscopy) to the macroscopic whole tumoral level (e.g., near infrared diffuse optical tomography, phase array detection, or reflectance imaging). The methods of the present invention may also be used to image dose responses.

Although this example is focused on MMP, and in particular, an MMP inhibitor, it will be appreciated that any enzyme inhibitors can be evaluated with the compositions and methods of the present invention. The following list sets forth potential candidates.

A. Broad Spectrum and Selective MMP Inhibitors

BB-2516 (marimastat)
BB-3644
BB-94 (batimastat)
BAY 12-9566
BMS-275291
CGS 27023 A Novartis
Chiroscience D2163
Chiroscience D1927
Chiroscience D5410
Cyclic peptides with HWGF motif (Nat Biotech 1999; 17:768-774)
CT-1746
Tissue inhibitors of metalloproteinases (TIMP)
Hydroxamates
Metastat (CollaGenex)
Neovastat (Aetema)
Non-hydroxamatic zinc binding molecules
Phenanthroline
Ro 32-3555 Roche
RS 130830 Roche Bioscience
Zinc chelators
Antisense nucleic acids
   139 individual compounds listed on pages 2743-2751 in Whittaker M. et al. Design and Therapeutic application of matrix metalloproteinase inhibitors. Chem. Rev., 1999; 99:2735-2776
MMP inhibitors in Brown et al, JACS, 2000, 122, 6799.
   B. Cathepsin B Inhibitors
Mu-Phe-homoPhe-fluoromethylketone (FMK)
peptidyl diazomethanes
E-64
CA-074 and other compounds (Chemistry & Biology, 2000, 7, 27)
CA-074-Me
Epoxide inhibitor (Chemistry & Biology, 2000, 7, 569)
   C. Cysteine Protease Inhibitor
Otto and Schirmeister, Chem. Rev., 1997, 97,133-171
   D. Cathepsin D
Pepstatin A (Leto et al., In Vivo, 1994, 8, 231-6)
   E. Other Enzyme Inhibitors
Caspase inhibitor
Protease inhibitor
Kinase inhibitor
Receptor Tyrosine Kinase Inhibitors
Phosphatase inhibitor
   F. Other Combinations

Any of the above in any combination as well as combined with cytostatic or other drug regimen e.g., gemcitabine, vinblastine, etc. (see, e.g., Cancer Res., 2000, 60:3207-3211).

### Other Embodiments

It is to be understood that while the invention has been described in conjunction with the detailed description thereof, the foregoing description is intended to illustrate and not limit the scope of the invention, which is defined by the scope of the appended claims. Other aspects, advantages, and modifications are within the scope of the following claims.

1. An activatable imaging probe comprising a chromophore attachment moiety and one or more chromophores, wherein the chromophores are chemically linked to the chromophore attachment moiety so that upon activation of the imaging probe, the optical properties of the chromophores are altered, wherein the imaging probe is activated by phosphorylation, dephosphorylation, pH mediated cleavage, conformation change, enzyme-mediated splicing, enzyme-mediated transfer of the one or more chromophores, hybridization of a nucleic acid sequence to a complementary target nucleic acid, binding of the probe to an analyte, chemical modification of the chromophore, or binding of the probe to a receptor.

2. The probe of claim 1, wherein the optical properties of the chromophores are altered by dequenching, quenching, changes in wavelength, changes in fluorescence lifetime, changes in spectral properties, or changes in polarity or combinations thereof.

3. The probe of claim 1, wherein the chromophores are selected from the group consisting of fluorochromes, non-fluorescent chromophores, fluorescence quenchers, absorption chromophores, and combinations thereof.

4. An activatable imaging probe comprising a chromophore attachment moiety and one or more chromophores, wherein the chromophores are chemically linked to the chromophore attachment moiety so that upon activation of the imaging probe the optical properties of the chromophores are altered, wherein the probe is activated by phosphorylation or dephosphorylation of the probe.

5. The probe of claim 4, wherein the phosphorylation is mediated by a kinase.

6. The probe of claim 4, wherein the dephosphorylation is mediated by a phosphatase.

7. The probe of claim 4, wherein the probe comprises one or more phophorylation sites.

8. The probe of claim 7, wherein the chromophore attachment moiety comprises the one or more phosphorylation sites.

9. The probe of claim 7, wherein the one or more phosphorylation sites are within a spacer between the chromophore attachment moiety and the chromophores.

10. An activatable imaging probe comprising a chromophore attachment moiety and one or more chromophores, wherein the chromophores are chemically linked to the chromophore attachment moiety so that upon activation of the imaging probe the optical properties of chromophores are altered, wherein the probe is activated by receptor-mediated binding.

11. An imaging probe comprising a chromophore attachment moiety and one or more chromophores, wherein chromophores are chemically linked to the chromophore attachment moiety so that upon activation of the imaging probe the optical properties of the chromophores are altered, wherein the probe contains a receptor polypeptide specific for ligand binding.

12. The probe of claim 11, wherein the chromophore attachment moiety comprises the receptor polypeptide.

13. The probe of claim 11, wherein the receptor polypeptide is within a spacer between the chromophore attachment moiety and the chromophores.

14. An activatable imaging probe comprising a chromophore attachment moiety, a functional group, and one or more chromophores, wherein the chromophores are chemically linked to the chromophore attachment moiety so that upon activation of the probe the optical properties of the chromophores are altered, wherein the probe is activated by enzyme-mediated removal of the functional group from the probe.

15. The probe of claim 14, wherein the functional group is chemically linked to the chromophore attachment moiety.

16. The probe of claim 14, wherein the functional group is chemically linked to a spacer between the chromophore attachment moiety and the chromophores.

17. An activatable imaging probe comprising a chromophore attachment moiety and one or more chromophores, wherein chromophores are chemically linked to the chromophore attachment moiety so that upon activation of the imaging probe the optical properties of chromophores are altered, wherein the probe is activated by enzyme-mediated splicing.

18. The probe of claim 17, wherein the probe comprises a nucleic acid sequence specific for enzyme-mediated splicing.

19. The probe of claim 18, wherein the chromophore attachment moiety comprises the nucleic acid sequence specific for enzyme-mediated splicing.

20. The probe of claim 18, wherein the nucleic acid sequence specific for enzyme-mediated splicing is within a spacer between the chromophore attachment moiety and the chromophores.

21. The probe of claim 1, wherein the probe is activated by enzyme-mediated transfer of a chromophore.

22. An activatable imaging probe comprising a chromophore attachment moiety and one or more chromophores, wherein chromophores are chemically linked to the chromophore attachment moiety so that upon activation of the imaging probe the optical properties of chromophores are altered, wherein the probe contains a nucleic acid sequence specific for a recombinase.

23. The probe of claim 22, wherein the chromophore attachment moiety comprises the nucleic acid sequence specific for a recombinase.

24. The probe of claim 22, wherein the nucleic acid sequence specific for a recombinase is within a spacer between the chromophore attachment moiety and the chromophores.

25. An activatable imaging probe comprising a chromophore attachment moiety and one or more chromophores, wherein the chromophores are chemically linked to the chromophore attachment moiety so that upon activation of the imaging probe the optical properties of the plurality of chromophores are altered, wherein the probe contains a transmembrane signal sequence.

26. The probe of claim 25, wherein the chromophore attachment moiety comprises the transmembrane signal sequence.

27. The probe of claim 25, wherein the transmembrane signal sequence is derived from a TAT protein comprising a caspase-3 sensitive cleavage site.

28. The probe of claim 25, wherein the transmembrane signal sequence is Gly-Arg-Lys-Lys-Arg-Gln-Arg-Arg (SEQ ID NO:15) or Gly-Arg-Lys-Lys-Arg-Arg-Gln-Arg-Arg (SEQ ID NO:16).

29. The probe of claim 1, wherein the probe is activated by hybridization of a nucleic acid sequence to a complementary target nucleic acid.

30. The probe of claim 29, wherein the chromophore attachment moiety comprises the nucleic acid sequence.

31. The probe of claim 29, wherein the nucleic acid sequence is within a spacer between the chromophore attachment moiety and the chromophore.

32. The probe of claim 1, wherein activation occurs upon binding of the probe to an analyte.

33. The probe of claim 32, wherein the analyte is H⁺, Ca²⁺, Na⁺, Mg²⁺, Mn²⁺, Cl⁻, Zn²⁺, O₂, NO, Fe²⁺, K⁺, or H₂O₂.

34. A method of in vivo optical imaging of a target in a subject, the method comprising:
(a) delivering to the subject an imaging probe of claim 1;
(b) allowing adequate time for the imaging probe to be activated within the target;
(c) illuminating the target with light of a wavelength absorbable by the chromophores;
(d) detecting a signal emitted by the chromophores; and
(e) forming an optical image from the emitted signal.

35. The method of claim 34, wherein steps (a)-(d) are repeated at predetermined intervals to enable evaluation of the emitted signal of the chromophores in the subject over time.

36. The method of claim 34, wherein the method is used to detect a disease in the subject.

37. The method of claim 34, wherein the method is used to characterize a phenotype or genotype of a disease in the subject.

38. The method of claim 34, wherein the method is used to characterize the severity of a disease.

39. The method of claim 36, wherein the disease is selected from the group consisting of cancer, cardiovascular diseases, neurodegenerative diseases, immunologic diseases, autoimmune diseases, inherited diseases, infectious diseases, bone diseases, and environmental diseases.

40. An in vivo optical imaging method for the simultaneous imaging of two or more different targets in a subject, the method comprising:
(a) delivering to a subject two or more different imaging probes of claim 1, each probe comprising a chromophore attachment moiety and one or more chromophores whose emitted signals are distinguishable from the one or more chromophores on each other probe;
(b) allowing adequate time for molecules in the two or more targets to activate the imaging probes;
(c) illuminating the target with light of one or more wavelengths absorbable by the chromophores;
(d) detecting signals emitted by the chromophores; and
(e) forming an optical image of the two or more different targets from the emitted signals.

41. The method of claim 40, wherein steps (a)-(d) are repeated at predetermined intervals to enable evaluation of the emitted signal of the chromophores from the two or more probes in the subject over time.

42. An optical imaging method for assessing activity of an agent in a subject, the method comprising:
(a) administering to the subject an imaging probe of claim 1;
(b) allowing time for a molecule in a target tissue to activate the probe, if the molecule is present;
(c) illuminating the target tissue with light of a wavelength absorbable by the chromophores;
(d) detecting a signal emitted by the chromophores;
(e) forming an optical image from the emitted signal;
(f) administering to the subject the agent and repeating steps (a)-(e); and
(g) comparing the emitted signals and images of steps (d) and (e) over time or at a different agent dose to assess activity of the agent.

43. A method for determining the presence of a composition in a subject, the method comprising:
(a) administering to a subject an imaging probe of claim 1, wherein activation occurs in the presence of the composition;
(b) allowing time for the composition in a target to activate the probe, if the composition is present;
(c) illuminating the target with light of a wavelength absorbable by the chromophores; and
(d) detecting a signal emitted by the chromophores, wherein a signal indicates the composition is present.

44. The method of claim 43, wherein the composition is a drug.

45. The method of claim 43, wherein the composition is a polypeptide expressed by a gene.

46. The method of claim 43, wherein steps (a)-(d) are repeated at predetermined intervals to enable the evaluation of the emitted signal of the chromophores in the subject over time.

47. A method for assessing the effective dosage of an agent in a subject, the method comprising:
(a) administering to the subject the agent at a specific dosage;
(b) administering to the subject an imaging probe of claim 1;
(c) allowing time for a molecule in a tissue of the subject to activate the probe, if the molecule is present;
(d) illuminating the tissue with light of a wavelength absorbable by the chromophores; and
(e) detecting the signal emitted from the chromophores to assess whether the specific dosage was effective.

48. The method of claim 47, wherein steps (a)-(e) are repeated at a different specific dosage and the detected signals from step (e) are compared to determine which dosage is most effective.

49. An optical imaging method for guiding therapeutic interventions in a subject, the method comprising:
(a) administering to the subject an imaging probe of claim 1;
(b) allowing time for a molecule in a tissue of the subject to activate the probe, if the molecule is present;
(c) illuminating the tissue with light of a wavelength absorbable by the chromophores; and
(d) detecting and using a signal emitted from the chromophores to guide a therapeutic intervention.

50. The method of claim 49, wherein the therapeutic intervention is surgical intervention.

51. The method of claim 34, wherein the subject is a mammal.

52. The method of claim 34, wherein the subject is a human.

53. The method of claim 34, wherein the subject is an animal model of disease.

54. The method of claim 34, wherein the illuminating and detecting steps are done using an endoscope, a catheter, a tomographic system, surgical goggles with attached bandpass filters, or an intraoperative microscope.

55. An in vitro optical imaging method for assessing activity of an agent in a sample, the method comprising:
(a) administering to the sample an imaging probe of claim 1;
(b) allowing time for a molecule in the sample to activate the probe, if the molecule is present;
(c) illuminating the sample with light of a wavelength absorbable by the chromophores;
(d) detecting a signal emitted from the chromophores;
(e) forming an optical image from the emitted signal;
(f) administering to the sample the agent and repeating steps (a)-(e); and
(g) comparing the emitted signals and images of steps (d) and (e) over time or at different agent doses to assess the activity of the agent.
